(19) United States
(12) Patent Application Publication
(10) Pub. No.: US 2009/0325931 A1
(43) Pub. Date: Dec. 31, 2009

(54) USE OF CDK INHIBITORS FOR THE TREATMENT OF GRANULOCYTE MEDIATED DISORDERS

(75) Inventors: Adriano Rossi, Edinburgh (GB); Christopher Haslett, Edinburgh (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: University Court of the University of Edinburgh, Edinburgh (GB)

(21) Appl. No.: 12/309,744

(22) PCT Filed: Jul. 30, 2007
(86) PCT No.: PCT/GB2007/002903

§ 371 Date: Jun. 18, 2009

(30) Foreign Application Priority Data
Jul. 28, 2006 (GB) 0615039.5
Aug. 25, 2006 (GB) 0616871.0

(51) Int. Cl. A61K 31/55; A61K 31/52; C12N 5/00; A61P 29/00; A61P 11/00

(52) U.S. Cl. 514/215; 514/263.4; 435/375

(57) ABSTRACT

Described herein is the use of CDK inhibitors such as roscovitine for inducing apoptosis of granulocytes, for example neutrophils. Their use for treating inflammatory diseases is also provided.

### FIELD OF THE INVENTION

The present invention relates to methods of inducing apoptosis in leukocytes and the use of such methods in the treatment of inflammatory diseases, in particular chronic and/or persistent inflammatory disease.

### BACKGROUND TO THE INVENTION

Neutrophilic granulocytes play a vital role in innate immunity and are rapidly recruited to sites of infection and injury. However, many defense mechanisms (e.g., release of granules contents and production of reactive oxygen species) used by these cells to kill, destroy and digest invading microorganisms are potentially deleterious to host tissue. Thus, it is vital that once the physiological function of these inflammatory cells has been achieved they are rapidly cleared from the inflammatory site. During spontaneously resolving inflammation, neutrophils undergo apoptosis; a pre-programmed and highly regulated cell death process (Savill, J. S. et al. J. Clin. Invest 83, 865-875 (1989) and Savill, J. Nature 343, 170-173 (1990)). Neutrophil survival and apoptosis are profoundly influenced by the inflammatory milieu; for example inflammatory mediators (e.g. GM-CSF, LPS), environmental conditions (e.g., hypoxia) and the presence of pro-apoptotic stimuli (e.g., TNFα, FasL) can dramatically alter neutrophil longevity (Gilroy, D. W. Nat. Rev. Drug Discov. 3, 401-416 (2004), Riley, N. A. et al. Anti-Inflammatory & Anti-Allergy Agents in Medicinal Chemistry 5, 3-12 (2006)). Once apoptosis has been engaged, neutrophil secretory activity is shut-down, the cells remain intact and are phagocytosed by macrophages employing novel recognition mechanisms that fail to elicit a pro-inflammatory response (Savill, J. S. et al. J. Clin. Invest 83, 865-875 (1989), Whyte, M. K. J. Immunol. 150, 5124-5134 (1993)). However, where macrophage phagocytosis or neutrophil apoptosis is impaired, chronic inflammation may ensue (Gilroy, D. W. Nat. Rev. Drug Discov. 3, 401-416 (2004), Riley, N. A. et al. Anti-Inflammatory & Anti-Allergy Agents in Medicinal Chemistry 5, 3-12 (2006) and Jonsson, H. Nat. Med. 11, 666-671 (2005)). Consequently, the mechanisms involved in regulating the balance between inflammatory cell survival and apoptosis is the subject of considerable research endeavor.

Cell division of eukaryotic cells occurs in four separate phases; G1 phase where DNA replication is prepared prior to the synthesis (S) phase, where chromosomal replication occurs; this is followed by a gap (G2) phase which prepares the cell before the mitosis (M) phase, ultimately leading to cell division before returning to G1 to repeat the cycle. In certain circumstances where, for example, growth factors are withdrawn or the cell is terminally differentiated as in neutrophils, the cell will rest in G0 phase. At each stage of the cell cycle, there are critical checkpoints that control the precise chronology and quality of the chromosomal replication and division before committing the cell to divide.

The cyclin-dependent kinases (CDKs) have traditionally been described as key regulators of the cell cycle, where different CDKs become activated during cell cycle progression when complexed with their associated cyclin partners (Vermeulen, K et al. Cell Prolif. 36, 131-149 (2003)). For this reason, inhibition of CDKs, by specific inhibitors, has been targeted in order to prevent or limit tumor progression. Indeed, CDK inhibitors are currently in clinical trials for esophageal, lung, prostate and non-small cell lung cancers (Senderowicz, A. M. Oncogene 22, 6609-6620 (2003)). Interestingly, CDKs can also regulate apoptosis and recent evidence suggests that these kinases may also play a defined role in the regulation of death in terminally differentiated neurons (Monaco & Vallano. Curr. Med. Chem. 10, 367-379 (2003)). Neutrophils, as like other granulocytes, are terminally differentiated cells and therefore, based on the current literature, CDK inhibitors such as R-roscovitine would be predicted to either have no effect, or similar to the effect on neurons, inhibit apoptosis.

### SUMMARY OF THE INVENTION

The present inventors have investigated the effect of CDK inhibitors on neutrophils and neutrophilic inflammation in vivo. As described above, given that granulocytes such as neutrophils are terminally differentiated cells and given the effect of CDK inhibitors on neurons, it was expected that CDK inhibitors would have either no effect or an inhibitory effect on apoptosis of these cells. However, as detailed below, contrary to expectations, the present inventors have shown that neutrophils express CDKs and that various CDK inhibitors directly induced caspase-dependent neutrophil apoptosis and inhibit survival induced by a plethora of survival factors.

Accordingly, in a first aspect of the present invention, there is provided a method of inducing or accelerating apoptosis of granulocytes, said method comprising administering to said granulocytes an effective dose of a CDK inhibitor.

In one embodiment, the granulocytes are non-proliferating granulocytes.

The discovery that CDK inhibitors induce apoptosis in granulocytes enables the use of such compounds in the treatment of diseases or conditions in which activity of granulocytes contribute to the pathology of the disease. Accordingly, in a second aspect of the invention, there is provided a method of treating granulocyte mediated disease in an individual, said method comprising administering to said individual an effective dose of a CDK inhibitor.

As described in the examples, the inventors have shown that in vivo the CDK inhibitor, R-roscovitine, enhances dramatically the resolution of a carrageenan-elicited murine model of acute pleural inflammation and that the resolution is driven by a R-roscovitine-induced caspase-induced pro-apoptotic effect. The anti-inflammatory and pro-resolving effects on inflammation were also demonstrated in two further inflammation models. The findings suggest that CDK inhibitors may be used to promote resolution of inflammatory diseases.

Accordingly, in one embodiment of the present invention, the disease is an inflammatory disease.

A third aspect of the invention comprises the use of a CDK inhibitor in the preparation of a medicament for the treatment of a granulocyte mediated condition, for example an inflammatory disease.

A further aspect comprises a CDK inhibitor for use in the treatment of a granulocyte mediated condition, for example an inflammatory disease.

However, the method of the invention may be used to treat any granulocyte mediated disease. In one embodiment of the invention, the granulocyte mediated disease is a disease mediated by non-proliferating granulocytes. In one embodiment, the disease is a neutrophil mediated disease. However, other embodiments of the invention may involve treatment of eosinophil or basophil mediated disease.

In a particular embodiment, the invention may be used to treat a disease for which one or more symptoms are principally caused by the presence, infiltration and/or activation of granulocytes. In one embodiment, the disease is a granulocyte mediated disease for which the presence, infiltration and/or activation of granulocytes is solely responsible for one or more symptoms.

In one embodiment, the disease or disorder is a non-neoplastic inflammatory disease.

In one embodiment, the disease is a non-proliferative stage of an inflammatory disease.

The disease may be a chronic disease. In another embodiment, the disease may be acute.

In the invention, any suitable CDK inhibitor may be used. In one embodiment of the invention, the CDK inhibitor is an inhibitor of CDK1 and/or CDK2. In another embodiment of the invention, the CDK inhibitor is an inhibitor of CDK5.

In one embodiment of the invention, the CDK inhibitor is a purine or pyrimidine analog. In a particular embodiment, the CDK inhibitor is roscovitine. In another embodiment, the CDK inhibitor is NG75. In another embodiment, the CDK inhibitor is hymenialdisine (HD).

Preferred features of each aspect of the invention are as for each of the other aspects mutatis mutandis unless the context demands otherwise.

### DETAILED DESCRIPTION

The present invention is based on the surprising demonstration that a variety of CDK inhibitors induce and accelerate apoptosis of granulocytes, for example of neutrophils, and thus such compounds may be used in the treatment of diseases associated with or mediated by granulocytes. As described above, although CDK inhibitors have been suggested for use in the treatment of various cancers, it was not expected that these compounds would have a pro-apoptotic effect on granulocytes. Indeed, based on the effects of CDK inhibitors on neurons and the fact that granulocytes are terminally differentiated cells, it would be expected that CDK inhibitors would inhibit apoptosis of granulocytes or would have no affect whatsoever on such cells.

The invention therefore provides a novel means of inducing apoptosis in granulocytes and represents a means to treat diseases mediated by granulocytes.

### Granulocytes

Granulocytes are a class of leukocytes characterized by prominent cytoplasmic granules. There are three major granulocyte cell types: neutrophils, eosinophils and basophils.

### Neutrophils

The most numerous of the granulocytes are the neutrophils which comprise approximately 60% of blood leukocytes. During inflammation the number of neutrophils present in the blood dramatically increases. These cells are highly phagocytic and form the first line of defense against invading pathogens, especially bacteria. They are also involved in the phagocytosis of dead tissue after injury during acute inflammation. Many of the defense mechanisms employed by neutrophils against pathogens, such as the release of granule contents and the generation of reactive oxygen species are pro-inflammatory and damaging to host tissue. In conditions characterized by excessive activation of neutrophils and/or impaired neutrophil apoptosis, chronic or persistent inflammation may result.

### Eosinophils

Eosinophils comprise approximately 1-3% of blood leukocytes. Their primary role is in defense against parasites, in particular against helminthes and protozoal infection. In this regard, the cells comprise lysosomal granules containing cytotoxic compounds such as eosinophil cation protein, major basic protein, and peroxidase and other lysomal enzymes. Eosinophils are attracted by substances released by activated lymphocytes and mast cells. Although eosinophils may play a role in regulating hypersensitivity reactions by, for example, inhibiting mast cell histamine release degranulation, these cells may also damage tissue in allergic reactions. The cells accumulate in tissues and blood in a number of circumstances, for example, in hayfever, asthma, eczema etc. As a result, through degranulation, they may contribute to or cause tissue damage associated with allergic reactions, for example in asthma or allergic contact dermatitis.

### Basophils

Basophils, which comprise less than 1% of circulating leukocytes, have deep blue granules that contain vasoactive substance and heparin. In allergic reactions, they are activated to degranulate, which may cause local tissue reactions and symptoms associated with acute hypersensitivity reactions.

### Treatment

The present invention may be used to treat any disease in which granulocytes contribute to the disease pathology. In one embodiment the disease is s a disease in which granulocytes are principally responsible for the disease pathology. Such diseases include, but are not limited to those characterised by leukocytosis, neutrophilia, granulocytosis, or eosinophilia. Such conditions may result in symptoms such as inflammation, allergic reactions, drug reactions, cardiac abnormalities etc.

Diseases for which the invention may find use include those mediated by neutrophils, eosinophils, basophils or two or more thereof.

In the context of the present invention, the terms “granulocyte mediated disease”, “granulocyte mediated inflammation” and “inflammatory disease” do not encompass neoplastic diseases. In one embodiment, the disease is a disease which is not caused by proliferation of leukocytes, for example by abnormally excessive production of leukocytes.

In the context of the present invention, “treatment” includes any regime that can benefit a human or non-human animal. The treatment may be in respect of an existing condition or may be prophylactic (preventative treatment). Thus, treatment may include curative, alleviation and/or prophylactic effects.

In one embodiment of the method of the invention, the method is a method of treating a non-proliferative stage of a granulocyte mediated disease. In one embodiment of the invention, the granulocyte mediated condition is a neutrophil mediated condition. Neutrophil mediated conditions for which the present invention may find use include, but are not limited to, neutrophil mediated inflammatory conditions such as arthritis, pleurisy, lung fibrosis, systemic sclerosis and chronic obstructive pulmonary disease (COPD).

In one embodiment of the invention, the neutrophil mediated condition is an inflammatory condition of the lung, for example pleurisy.

In another embodiment, the neutrophil mediated condition is pulmonary fibrosis or chronic obstructive pulmonary disease (COPD).

In a further embodiment the neutrophil mediated disease is rheumatoid arthritis, systemic sclerosis or chronic obstructive pulmonary disease (COPD).

In another embodiment of the invention, the granulocyte mediated condition is an eosinophil mediated condition. Eosinophil mediated conditions for which the present invention may find use include, but are not limited to inflammatory lung disease, for example, asthma, atopic dermatitis, NERDS (nodules eosinophilia, rheumatism, dermatitis and swelling), hyper-eosinophilic syndrome or pulmonary fibrosis, contact dermatitis, eczema, hayfever or other allergic reactions. Other conditions, in which eosinophils may be involved and for which the invention may be used include inflammatory bowel disease (IBD), vasculitic granulomatous diseases including polyarteritis and Wegeners granulomatosis, autoimmune diseases, eosinophilic pneumonia, sarcoiditis and idiopathic pulmonary fibrosis.

In a further embodiment of the invention, the granulocyte mediated condition is a basophil mediated condition for example an allergic reaction, such as an acute hypersensitivity reaction. Other basophil mediated conditions for which the present invention may find use include, but are not limited to, asthma and allergies such as hayfever, chronic urticaria, psoriasis, eczema, inflammatory bowel disease, ulcerative colitis, Crohn's disease, COPD (chronic obstructive pulmonary disease) and arthritis.

Thus, diseases and conditions which may be treated by the present invention include diseases of different tissues and organs.

For example, in one embodiment, the invention may be used in the treatment of a disease or diseases of the respiratory system. For example, the invention may be used to treat inflammatory conditions of the lung, such as interstitial lung diseases. Interstitial lung diseases, which may be treated using the invention, include both acute diseases, such as acute interstitial pneumonia, and chronic conditions, such as idiopathic pulmonary fibrosis. Other respiratory diseases and conditions which may be treated using the methods of the invention include pleurisy, asthma, in particular severe asthma and steroid resistant asthma, chronic obstructive pulmonary disease (COPD), and acute respiratory disease.

The invention may also be used to treat diseases/conditions of the heart such as reperfusion damage caused by myocardial infarction, atheroma;

The invention may also be used to treat diseases/conditions of the kidney such as glomerular nephritis;

The invention may also be used to treat diseases/conditions of the skin such as acne, psoriasis, or eczema

Other conditions for which the invention may find use include arthritis, in particular rheumatoid arthritis, gout, secondary brain stem haemorrhage and stroke.

In one embodiment, the disease/condition is an allergic condition. In one such embodiment, the allergen is non-self i.e. not an autoimmune reaction.

In one particular embodiment of the invention, the diseases or disorder is pleurisy.

In another particular embodiment of the invention, the diseases or disorder is pulmonary fibrosis.

In another particular embodiment of the invention, the disease or disorder is arthritis.

### CDK Inhibitors

In the methods of the invention, any suitable inhibitor may be used. CDK inhibitors which may be used include, but are not limited to, purine and pyrimidine derivatives such as olomouscine, roscovitine, R-roscovitine (seliciclib), N⁹-isopropyl-olmoucine or NG75 and hymenialdisine. Other purine-based CDK inhibitors which may be used include CGP79807 and CGP74514 (which has cyclichydroxy or amino-alkyl amino groups present as C2 respectively, Imbach et al, Bioorg. Med. Chem. Lett., 9, 91-96 (1999); Dreyer et al, J. Med. Chem., 44, 524-530 (2001)), guanine derivatives such as NU2058 and NU6102 (Arris et al, J. Med. Chem., 43, 2797-2804 (2000), (Davies et al, Nat. Struct. Biol., 9, 745-749 (2002), phenylaminopyridines such as CGP60474 (Furet et al, J. Comput. Aided Mol. Des., 14, 403-409 (2000), CINK4 (Soni et al, J. Nat. Cancer Inst., 93, 436-446 (2001), NU6027, thiazolopyrimidines (Fischer et al, Eur. J. Cancer, 38 (Suppl. 7), 124 (2002). Other CDK inhibitors which may be used include purvalanols, such as purvalanol A and purvalanol B, indirubin, oxindole based CDK inhibitors, (Kent et al, Biochem. Biophys. Res. Commun. 260, 768-774 (1999) such as phenylhydrazone oxindole and anilinomethylene oxindole, indenopyrazoles, (Nuglel et al, J. Med. Chem., 44, 1334-1336 (2001). Other CDK inhibitors which may be used include flavopiridol and analogs thereof, such as 2-benzylidine-benzo-furan-3-ones (Kim et al J. Med. Chem., 43, 4126-4134 (2000); Schoepfer et al. J. Med. Chem., 45 1741-1747 (2002)). staurosporine, and analogues thereof, for example 7-hydroxystaurosporine, bryostatin-1, BMS-387032, SU9516, AZ703, E7070, amino imidazopyridine 1d, NU 6140, flavopiridol, AG-024322, PD-0332991, PNU-252808, diarylureas, and paullones, such as kenpaullone, alsterpaullone, butyrolachtone-1, sangivamychin, SU9516 AZ703. Details of many of these inhibitors are described in J. Clin. Oncol. 23(36) 9408-9421 and J. Clin. Oncol. 24(11) 1170-1783. Further CDK inhibitors, which may be used in the present invention, are described in WO 01/44217, WO 99/24416, WO 01/44242, WO97/20842, WO98/05335, and WO99/07705.

In one embodiment, the CDK inhibitor is roscovitine (6-benzylamino-2-[(R)-1-ethyl-2-hydroxyethylamino]-9-isopropylpurine).

In another embodiment, the CDK inhibitor is NG75.

In a further embodiment, the CDK inhibitor is hymenialdisine (HD).

Further, in these methods, treatment with combinations of the CDK inhibitors described herein with other agents useful for treating the disorders is provided. The choice of other agent will depend on the particular condition being treated and will be at the discretion of the physician.

For example, other agents which may be used in combination with CDK inhibitors in the invention include but are not limited to NSAIDs, glucocorticosteroids, disease-modifying antirheumatic drugs (DMARDs—e.g., intramuscular gold, hydroxychloroquine, sulphasalazine and methotrexate—for arthritis) and anti-TNF therapy with biologics.

Where the CDK inhibitors are to be used for treatment in combination with one or more other agents, the concentration of the CDK inhibitor(s) and the other agent(s) is preferably provided at concentrations sufficient to provide a synergistic effect. Synergism is preferably defined as an RI of greater than unity using the method of Kern (Kern, D. H, et al. Cancer Res, 48:117-121, (1988)) as modified by Romaneli (Romanelli, S et al. Cancer Chemother Pharmacol, 41: 385-390, (1998)). The RI may be calculated as the ratio of expected cell survival (Sₑₓₚ, defined as the product of the survival observed with drug A alone and the survival observed with drug B alone) to the observed cell survival (S_{obs}) for the combination of A and B (RI=Sₑₓₚ/S_{obs}). Synergism may then be defined as an RI of greater than unity.

### Pharmaceutical Compositions

The CDK inhibitors for use in the invention may be administered as a pharmaceutical composition. Pharmaceutical compositions according to the present invention, and for use in accordance with the present invention, may comprise, in addition to active ingredients, a pharmaceutically acceptable excipient, a carrier, buffer, stabiliser or other materials well known to those skilled in the art (see, for example, Remington: the Science and Practice of Pharmacy, 21^{st} edition, Gennaro A R, et al, eds., Lippincott Williams & Wilkins, 2005). Such materials may include buffers such as acetate, Tris, phosphate, citrate, and other organic acids; antioxidants; preservatives; proteins, such as serum albumin, gelatin, or immunoglobulins; hydrophilic polymers such as polyvinylpyrrolidone; amino acids such as glycine, glutamine, asparagine, histidine, arginine, or lysine; carbohydrates; chelating agents; tonicifiers; and surfactants.

The pharmaceutical compositions may also contain one or more further active compounds selected as necessary for the particular indication being treated, preferably with complementary activities that do not adversely affect the activity of the CDK inhibitor. For example, in the treatment of inflammatory disease such as arthritis, in addition to CDK inhibitors, other anti-inflammatory agents, such as cyclooxygenase-2 (COX-2) inhibitors, may be used.

The active ingredients (e.g. CDK inhibitors and/or other anti-inflammatory agents) may be administered via microspheres, microcapsules, liposomes, other microparticulate delivery systems. For example, active ingredients may be entrapped within microcapsules which may be prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions. For further details, see Remington: the Science and Practice of Pharmacy, 21^{st} edition, Gennaro A R, et al, eds., Lippincott Williams & Wilkins, 2005.

Sustained-release preparations may be used for delivery of active agents. Suitable examples of sustained-release preparations include semi-permeable matrices of solid hydrophobic polymers containing an active agent, e.g. an antibody, which matrices are in the form of shaped articles, e.g. films, suppositories or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and ethyl-L glutamate, non-degradable ethylene-vinyl acetate, degradable lactic acid-glycolic acid copolymers, and poly-D-(−)-3-hydroxybutyric acid.

### Dose

The compositions of and for use in the invention are preferably administered to an individual in a “therapeutically effective amount”, this being sufficient to show benefit to the individual. The actual dosage regimen will depend on a number of factors including the condition being treated, its severity, the patient being treated, the agent(s) being used, and will be at the discretion of the physician.

The optimal dose can be determined by physicians based on a number of parameters including, for example, age, sex, weight, severity of the condition being treated, the active ingredient(s) being administered and the route of administration.

### BRIEF DESCRIPTION OF THE FIGURES

The invention will now be described further in the following non-limiting examples. Reference is made to the accompanying drawings in which:

FIG. 1(A) illustrates the effects of CDK inhibitors on human neutrophil apoptosis, as assessed by annexin-V-FITC binding. Values represent the mean±s.e.m. of at least n=3. **(p<0.001) denotes significantly different from control.

FIG. 1(B) illustrates that different CDK inhibitors induce time-dependent apoptosis of neutrophils. Values represent the mean±s.e.m. of at least n=3. *(p<0.05), **(p<0.001) denotes significantly different from control.

FIG. 1 (C-E) illustrate that the CDK inhibitor NG75 enhances annexin V binding and induced morphological changes indicative of neutrophil apoptosis. Flow cytometry dot plots of NG75 treated neutrophils, taken at time intervals of C) 0 h, D) 8 h, E) 20 h. Non-apoptotic cells are shown in grey, annexin-V-FITC +ve cells in green and annexin-V-FITC +ve/PI+ve cells in red. Micrographs of NG75 treated neutrophils, taken at time intervals of C) 0 h: Typical neutrophil morphology of multi-lobed nuclei. D) 8 h: Arrow indicates typically apoptotic neutrophil morphology, showing condensed nuclei. E) 20 h: Arrow indicates apoptotic neutrophil without nuclear staining and the cell membrane appears ‘leaky’. This type of morphology may account for the annexin-V+ve/PI+ve cells seen by flow cytometry at 20 h. Apoptosis was assessed by annexin-V-FITC binding, and checked by morphology.

FIG. 1 (F-H) illustrate CDK Inhibitors reverse dbcAMP, GMCSF and LPS mediated survival of neutrophils. (p<0.001) denotes significantly different from control of relevant survival agent. Values represent the mean±s.e.m. of n=3. # (p<0.05) denotes significant difference from control. *(p<0.05), **. (p<0.001) denotes significantly different from control of relevant survival agent.

FIG. 2 illustrates CDK protein and activity in neutrophils and mechanisms governing the pro-apoptotic effect of CDK inhibitors:

FIG. 2 (A) illustrates that the caspase inhibitor zVAD-fmk inhibits R-roscovitine induced apoptosis of neutrophils. Values represent the mean±s.e.m. of n=4. *(p<0.05) significant difference from control.

FIG. 2 (B) illustrates that CDK1 and CDK2 proteins are expressed in neutrophils and R-roscovitine induces caspase cleavage in neutrophils. Blots are representative of at least 3 separate experiments.

FIG. 2 (C) illustrates that CDK1 and CDK2 protein expression does not change during neutrophil apoptosis. Blots are representative of 3 separate experiments.

FIGS. 2 D and E illustrates that CDK1 activity is present in neutrophils and decreases following apoptosis induced by anti-Fas antibody CH11. Results are representative of at least 3 separate experiments. Results are representative of at least 3 separate experiments.

FIG. 2 (F) illustrates that R-roscovitine reduces survival factor induced Mcl-1 protein expression in neutrophils. Human neutrophils (5×10⁶ cells/ml) were treated with buffer, GM-CSF (50 U/ml), R-roscovitine (20 μM), or GM-CSF (50 U/ml) plus R-roscovitine (20 μM) for 4 h. The samples were subsequently lysed and the proteins separated on an SDS gel. Following western blotting, PVDF membranes were probed with anti-Mcl-1 antibody. Blots are representative of at least 3 separate experiments.

FIG. 3 illustrates the effect of the CDK inhibitor R-roscovitine on the resolution of carrageenan-induced pleurisy:

(A-C) The CDK inhibitor R-roscovitine dose-dependently promoted the resolution of inflammation in vivo. Number of total pleural inflammatory cell numbers (A), mononuclear cells and PMNs numbers (B) and exudate volumes (C) were measured. Values represent the mean±s.e.m. of 8-10 mice per group. **p<0.01 and ***p<0.001 denotes significant difference from DMSO control.

(D-F) The CDK inhibitor R-roscovitine reduced pro-inflammatory cytokines in vivo., IL-6 (D), IFN-γ (E) and MCP-1 (F) Values represent the mean±s.e.m. of 8-10 mice per group. **p<0.01 and ***p<0.001 denotes significant difference from DMSO control.

(G) The CDK inhibitor R-roscovitine promoted the resolution of inflammation in vivo in a time-dependent manner. 24 h following intra-pleural injection of 1% carrageenan, male C57/bl6 mice were treated with vehicle control (DMSO) or 100 mg/Kg R-roscovitine (i.p.). Mice were killed at either 24 h, 48 h and 166 h post-carrageenan and the total pleural inflammatory cell numbers determined. Values represent the mean±s.e.m. of five to six mice per group. **p<0.01 and ***p<0.001 denotes significant difference from DMSO control.

FIG. 4 illustrates the role of caspase dependent apoptosis in R-roscovitine enhanced resolution of carrageenan induced pleurisy: (A and B) The caspase inhibitor zVAD-fmk prevents R-roscovitine-induced resolution of carrageenan-induced inflammation. total pleural inflammatory cell numbers (A) and exudate volumes (B) were measured. Values represent the mean±s.e.m. of 8-10 mice per group. *p<0.05, **p<0.01 and ***p<0.001 denotes significant difference from DMSO control.

(C) The caspase inhibitor zVAD-fmk prevents apoptosis during R-roscovitine-induced resolution of carrageenan-induced inflammation. 24 h following intra-pleural injection of 1% carrageenan, male C57/bl6 mice were treated i.p. with 10 mg/Kg R-roscovitine (i.p.) and/or 1 μg/Kg zVAD-fmk (i.p. at 4 h intervals). All mice were killed 36 h post-carrageenan and apoptosis was analyzed in pleural inflammatory cells by annexin V labeling and flow cytometry. Inflammatory cell apoptosis was also assessed by morphology on haematoxylin and eosin stained cyto-centrifuge preparations. Values represent the mean±s.e.m. of 8-10 mice per group. *p<0.05 denotes significant difference from DMSO control.

(D-G) R-roscovitine reduces inflammation in pleural lavage exudates and lung tissue and decreases numbers of macrophages containing apoptotic bodies in pleural lavage. 24 h following intra-pleural injection of 1% carrageenan, male C57/bl6 mice were treated i.p. with vehicle control (D and F) and 100 mg/Kg R-roscovitine (i.p.) (E and G). All mice were killed 36 h post-carrageenan and cyto-centrifuge preparations and tissue sections of pleural lavage and lungs respectively were made and stained with haematoxylin and eosin analyzed microscopically by morphological examination (original magnification×40), where viable neutrophils are indicated with asterisks (D) and arrows indicate phagocytosed apoptotic neutrophils (E).

FIG. 5 illustrates the effect of the CDK inhibitor R-roscovitine on the resolution of bleomycin-induced lung inflammation and serum induced arthritis:

(A) The total number of neutrophils found in the lung is reduced by R-roscovitine following bleomycin-induced lung inflammation. Values represent the mean±s.e.m. of six mice per group. **p<0.01 denotes significant difference from DMSO control.

(B-D) R-roscovitine inhibits inflammation and tissue damage in a chronic model of bleomycin-induced lung injury. Images are representative of six mice per group.

(E) R-roscovitine enhances the resolution of passively-induced arthritis. Data are expressed as the percentage of the clinical score obtained on day 3 before the first injection of R-roscovitine. ***P<0.001 analyzed by two-way ANOVA.

### EXAMPLES

### Methods

Human neutrophil Isolation and Culture

Human neutrophils were isolated from freshly drawn venous blood and separated after citration, dextran sedimentation, and discontinuous PBS-Percoll gradients (Ward, C. et al. J. Bio. Chem. 274, 4309-4318 (1999), Haslett, C et al. Am. J. Pathol. 119, 101-110 (1985)) a process that takes circa 2 h to complete. Neutrophils (5×10⁶/ml) were then cultured for the indicated times at 37° C. in Iscove's modified Dulbecco's medium (IMDM) supplemented with 10% autologous serum. Experiments were carried out in triplicate and repeated at least 3 times.

### Assessment of Neutrophil Apoptosis

Following incubation with the reagents [R-roscovitine, (R)-2-[{9-(1-methylethyl)-6-[(phenylmethyl)amino]-9H-purin-2-yl]amino}-1-butanol (from A.G. Scientific, Inc), hymenialdisine HD, 4-(2-Amino-4-oxo-2-imidazolidin-5-ylidene)-2-bromo-4,5,6,7-tetrahydropyrrolo[2,3-c]azepin-8-one (a gift from Dr Laurent Meijer, Roscoff, France) and NG-75 (2-(1R-isopropyl-2-hydroxyethylamino)-6-(4-methoxy-benzylamino)-9-isopropylpurine, a gift from Dr Nathanael Gray, University of California, Berkeley, USA), db-cAMP (Sigma), GMCSF (R & D Systems) and LPS (E. Coli 0127:B8, Sigma)], 100 μl aliquots of neutrophils were cytocentrifuged, fixed in methanol and stained with Diff-Quik™ (Baxter Healthcare). Neutrophils were assessed by light microscopy for morphological changes characteristic of apoptosis (Savill, J. et al. J. Clin. Invest 83, 865-875 (1989), Ward, C. et al. J. Biol. Chem. 274, 4309-4318 (1999)). At least 500 cells were counted per slide and the percentage apoptosis calculated. Apoptosis and loss of membrane integrity was also assessed by flow cytometry using human recombinant annexin-V-FITC (Roche, UK) to measure phosphatidylserine (PS) exposure, in combination with propidium iodide (Sigma). Stock annexin-V-FITC was diluted at 1:500 in annexin-V binding buffer (500 ml Hanks Balanced Salt Solution containing 5 μM CaCl₂) and was added at a volume of 280 μl to 5×10⁴ cells, and then incubated on ice for 10 min. 1 μl of PI (stock 1 mg/ml) was added to the samples shortly before analysis using the EPICS XL2 flow cytometer (Beckman Coulter, High Wycombe, UK). Where apoptosis data are presented as annexin-V binding, apoptosis was also confirmed by morphological assessment.

### Western Blotting

Cells (5×10⁶) were washed in PBS before being resuspended in 100 μl of lysis buffer containing 1:100 Sigma protease inhibitor cocktail, 7 mM AEBSF, 3 mM aprotinin, 10 mM benzamidine, 10 mM β-glycerophosphate, 0.4 mM leupeptin, 40 mM levamisole, 30 mM pepstatin A, 20 mM sodium orthovanadate and 0.1% NP-40 in 1 ml TBS (Ward, C. et al. J. Biol. Chem. 274, 4309-4318 (1999)). Cells were then incubated on ice for 15 min. Samples were subsequently centrifuged at 23,100 gₐᵥ at 4° C. for 20 min. Protein (40 μg) or the equivalent of 1.5×10⁶ cells per lane was loaded and the samples resolved by SDS polyacrylamide Gel Electrophoresis (PAGE) and transfer to polyvinylidene difluoride (PVDF) membranes. Blots were then blocked with 5% skimmed milk powder in TBS/Tween before probing with anti-CDK1 (BD Transduction Laboratories), anti-CDK2 (BD Transduction Laboratories), anti-CDK5 (Santa-Cruz Biotechnology), anti-Caspase-3 (Cell Signaling Technologies), Mcl-1 (Santa-Cruz Biotechnology) or β-actin (Sigma) antibodies.

### Kinase Assays

Neutrophils (20×10⁶) were pelleted and stored at −80° C. until assayed. Kinase activity was assayed by immunoprecipitation of the kinase followed by incubation with histone H1 and [Y-³²P] ATP. The immunoprecipitations and histone H1 kinase assays were performed (Gil-Gomez, G et al. EMBO J. 17, 7209-7218 (1998)). The A17 antibody (Pharmingen) for CDK1 and the M2 antibody (Santa Cruz Biotechnology) were used for CDK2 immunoprecipitations. The kinase assays were resolved on an SDS-12% polyacrylamide gel, stained with Coomassie Blue to visualize the histone H1 bands, dried and exposed for autoradiography.

### Carrageenan-Induced Pleurisy

Male 8-12 week old C57BL/6J mice (B&K, Hull, UK) were housed in the University of Edinburgh Animal Facilities in accordance with local guidelines. Animals were fed on a normal diet with tap water ad libitum. Mice were injected intra-pleurally with 0.1 ml 1% λ-carrageenan (a gift from Marine Colloids, Philadelphia, Pa.). Mice were treated with either 0.5 ml saline control, 0.5 ml 0.5% DMSO vehicle control, 10 mg/Kg or 100 mg/Kg R-roscovitine (i.p.) 24 h after injection with carrageenan. Animals were culled by a rising concentration of CO₂ at various times after injection of carrageenan (36-166 h). Pleural cavities were washed with 1 ml of 3.15% (weight/volume) sodium citrate in phosphate buffered saline (PBS). Edema formation was measured by weighing the total pleural exudate and total cell counts were measured with a Coulter® Counter (model DN; Beckman Coulter, High Wycombe, UK).

Administration of zVAD-fmk in vivo

z-Val-Ala-DL-Asp-fluoromethylketone (zVAD-fmk) (Bachem (UK) Ltd, St Helens, UK) was dissolved in 100% DMSO at 200 mg/ml and diluted in 0.9% saline to a final concentration of 1 mg/ml. 24 h following the intra-pleural injection of 0.1 ml 1% λ-carrageenan, some animals were injected i.p. with 0.5 ml 10 mg/kg R-roscovitine and/or 0.5 ml 1 ug/kg zVAD-fmk. Two additional doses of zVAD-fmk were given i.p. 4 and 8 h later and all animals were killed with a rising concentration of CO₂ 12 h later. All control animals were treated with appropriate amounts of DMSO vehicle.

### Cytokine Analysis

Cell cytokine profiles were measured with a murine inflammation cytokine bead assay (Catalog No 552362; BD Bioscience, Oxford, UK) according to the manufacturer's instructions and analyzed by flow cytometry with the BD Facs Calibur (BD Bioscience, Oxford, UK).

Morphological Assessment of Apoptosis and Macrophage Phagocytosis of Apoptotic Cells from the Pleural Cavity.

Pleural cavities were washed with 1 ml of 3.15% (weight/volume) sodium citrate in phosphate buffered saline (PBS) and cells were centrifuged (300 g). Cells were resuspended at 1×10⁶ cells/ml, and cyto-centrifuge preparations were made and stained with Diff-Quik™ (Baxter Healthcare). Cells were examined microscopically to assess free apoptotic cells, the percentage of macrophage phagocytosis and the phagocytic index for each group.

### Bleomycin-Induced Lung Injury

Male 8-12 week old C57BL/6J mice (B&K, Hull, UK) were housed in the University of Edinburgh Animal Facilities in accordance with local guidelines. Animals were fed on a normal diet with tap water ad libitum.

Mice were given either 0.05 ml of 0.1 U bleomycin (Apollo Scientific, Bredbury, UK) or saline intra-tracheally (sham control) and then 24 h later were treated with either 0.5 ml of 0.5% DMSO vehicle control or 100 mg/Kg R-roscovitine. Some animals were left untreated to compare normal physiology. Animals were culled by a lethal dose of pentobarbitone 72 h or seven days after bleomycin or saline administration. In the acute 72 h experiments, bronchioalveolar lavages were performed with three sequential washes with 0.8 ml of ice-cold saline before perfusion with 4% formaldehyde for tissue histological analysis. Differential cell counts were performed on cytocentrifuge preparations with eosin and haemotoxylin staining. Histological analysis of the seven-day experiments were performed without bronchioalveolar lavages to maintain tissue integrity. Experiments were performed with six mice per group with untreated control, sham control (saline and DMSO treatment), R-roscovitine control (saline and 100 mg/Kg R-roscovitine), Bleomycin/vehicle (DMSO) control and Bleomycin/R-roscovitine treatment groups.

### Assessment of Lung Injury

Lung injury was assessed by histological examination where lungs were inflated and fixed with 1 ml 10% formalin and decalcified with 5% nitric acid for 3 h. The paraffin-embedded lungs were sectioned and stained with haematoxylin and eosin for morphological examination by light microscopy.

### Induction and Assessment Of Arthritis

Sera (100 μl) extracted from 60 day old arthritic K/B×N mice was injected intra-peritoneally into 6-8 week old female C57BL/6J mice (B&K, Hull, UK). Recipients received 2 injections 2 days apart and R-roscovitine or vehicle was administered i.p. as indicated in the figure legend. Arthritis was scored by clinical examination. Briefly, a score of 1 was given for erythema alone or swelling of 1 digit. A score of 2 resulted from erythema plus swelling of the tarsal joints or swelling of the hock or wrist joint alone. A maximum score of 3 was achieved when swelling was present in both tarsal and hock joints, or both wrist and digits, or more than 2 digits and the tarsal joints. The score for each limb was added giving a maximum score of 12 and the mean score reflects the total score divided by the number of recipient mice per group. Data are expressed as the percentage of the clinical score obtained on day 3 before the first injection of R-roscovitine.

### Statistical Analysis

Analysis of in vitro experiments was carried out using ANOVA and Student-Newman-Keuls comparison tests. P values of <0.05 were considered significant. Results are expressed as mean±s.e.m. of at least 3 separate experiments each performed in triplicate. All in vivo experiments were done with 6-9 mice per group, with some experiments repeated to verify the original findings. Statistical analysis was performed by a one-way ANOVA with Bonferroni multiple comparison post hoc test with a 95% confidence interval. Data are expressed as mean±s.e.m.

### Results

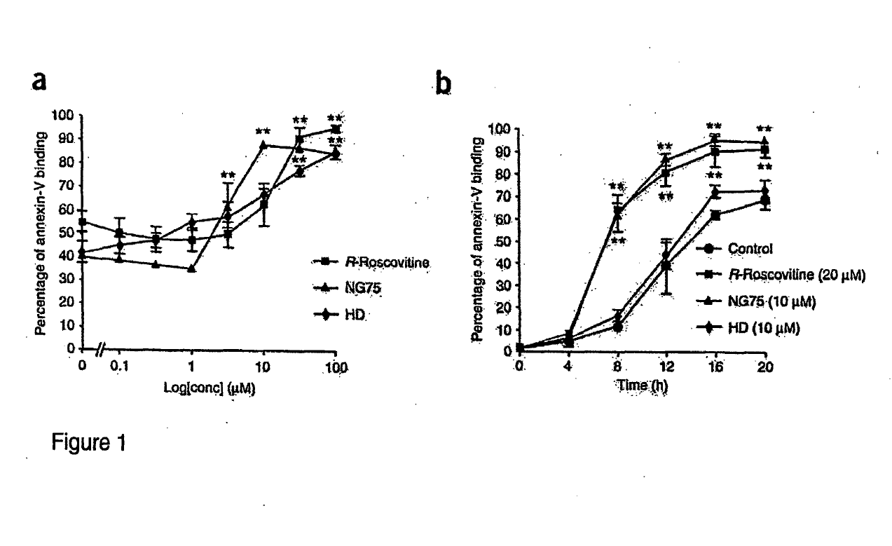

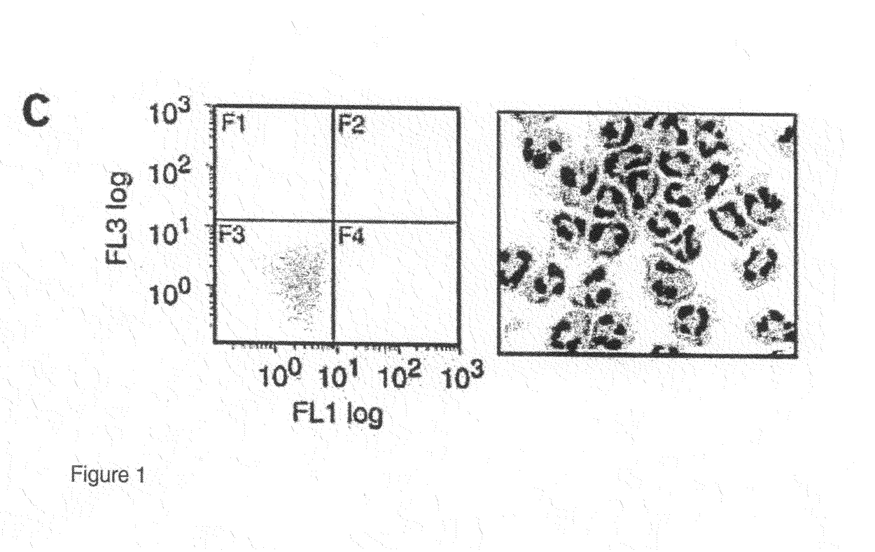

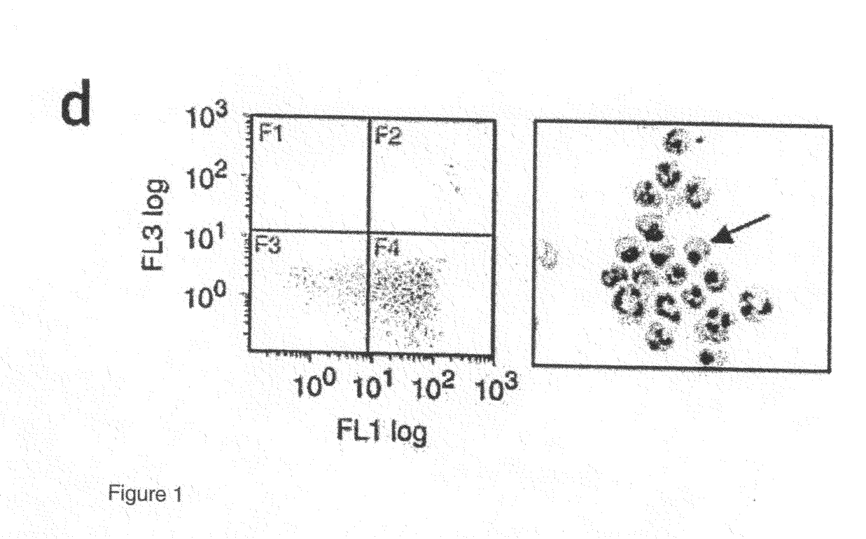

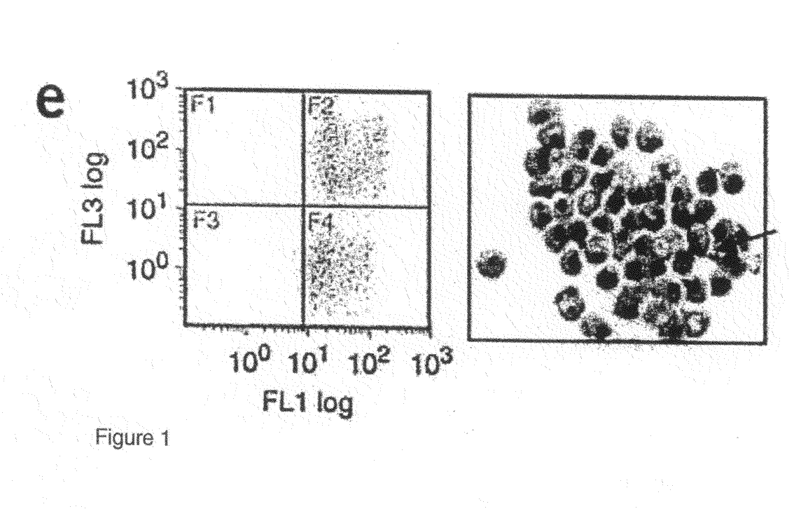

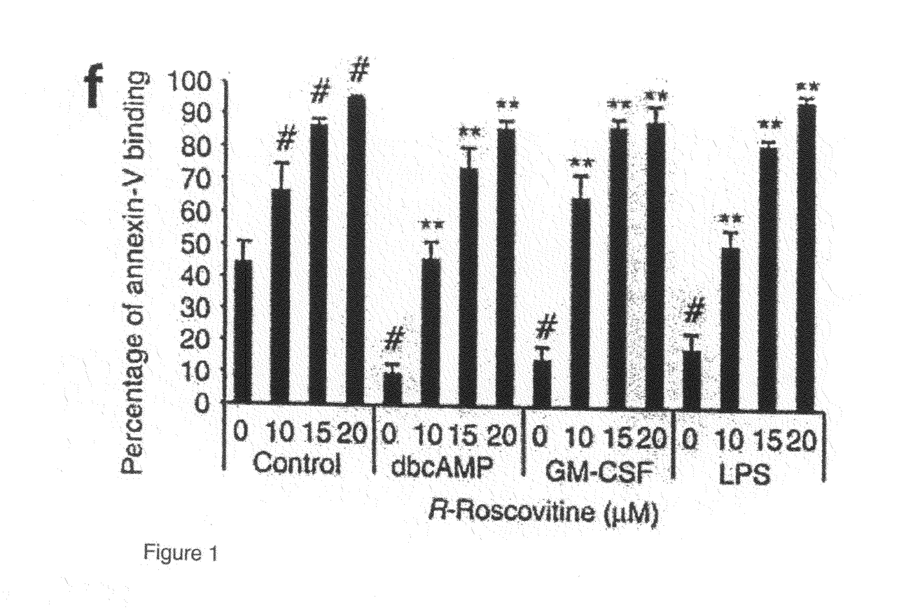

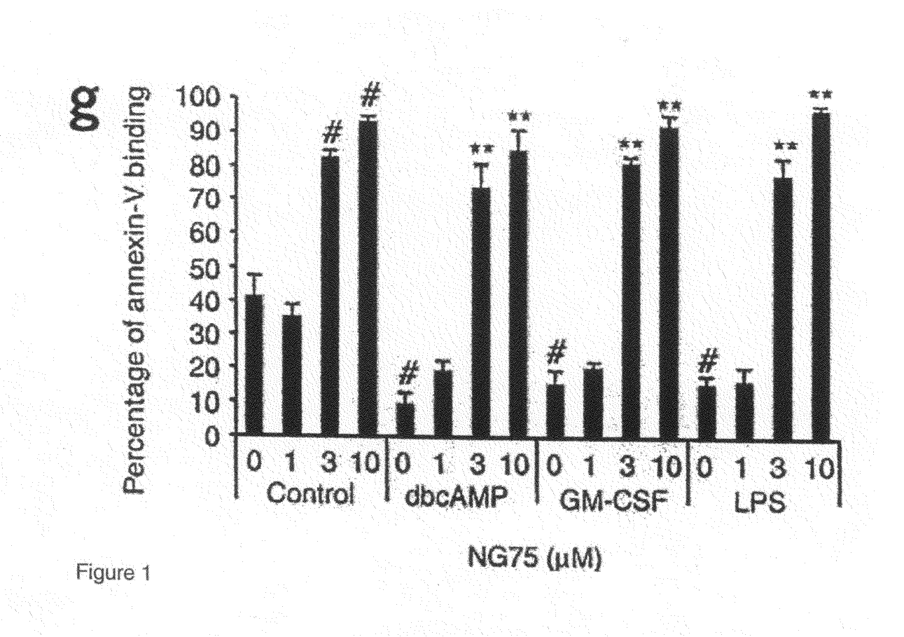

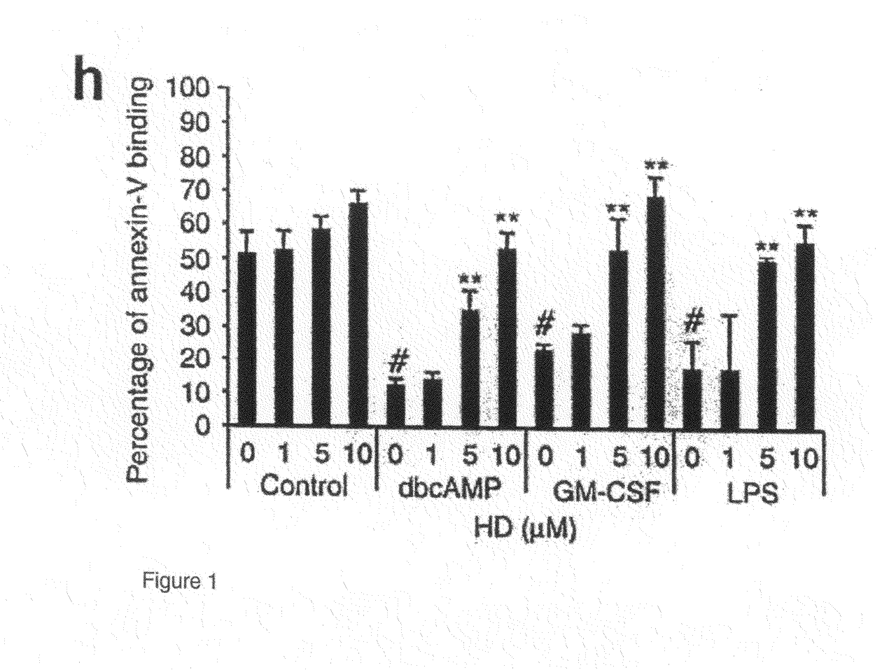

In order to investigate whether CDK inhibitors could affect neutrophil apoptosis directly, human neutrophils were incubated with increasing concentrations of structurally diverse CDK inhibitors (R-roscovitine (De Azevedo, W. F. et al. Eur. J. Biochem. 243, 518-526 (1997), Meijer, L. et al. Eur. J. Biochem. 243, 527-536 (1997), Bach, S. et al. J. Biol. Chem. 280, 31208-31219 (2005)), NG75 (Gray, N. S. et al. Science 281, 533-538 (1998), Chang, Y. T. et al. Chem. Biol. 6, 361-375 (1999)) and HD (Meijer, L. et al. Chem. Biol. 7, 51-63 (2000), Williams et al Eur. J. Immunol. 30, 709-713 (2000)) over a 20 h period. Notably, the concentrations of CDK inhibitors used were at, or below, those previously published to have a specific effect on CDK inhibition. Briefly, human neutrophils (5×10⁶ cells/ml) were incubated in IMDM with 10% autologous serum for 20 h, with increasing concentrations of R-roscovitine, NG75 or HD. Apoptosis was assessed by annexin-V-FITC binding, and confirmed by morphological assessment. Values represent the mean±s.e.m. of at least n=3. ** (p<0.001) denotes significantly different from control. The results are shown in FIG. 1 (A). FIG. 1(B) shows that different CDK inhibitors induce time-dependent apoptosis of neutrophils. Briefly, human neutrophils (5×10⁶ cells/ml) were incubated over 20 h in IMDM containing 10% autologous serum with 20 μM R-roscovitine, 10 μM NG75 or 10 μM HD. Apoptosis was assessed by annexin-V-FITC binding, and confirmed by morphological assessment.

As shown, there was a dramatic concentration—(FIG. 1A) and time-dependent (FIG. 1B) increase in neutrophil apoptosis induced by all three CDK inhibitors.

Typical annexin-V and PI profiles of neutrophil populations demonstrated that after 8 h of NG75 treatment a marked increase in annexin V positive cells were observed (FIG. 1C-E). With more prolonged treatments (i.e. 20 h) an increased number of annexin-V/PI positive cell populations were observed, corresponding to cells that have undergone secondary necrosis and can be identified morphologically by their nuclear loss and ruffled plasma membrane (FIG. 1E). Similar profiles were seen with R-roscovitine and HD treatment and all apoptosis data presented as % annexin V binding were confirmed by morphological assessment (data not shown). Thus R-roscovitine, NG75 and HD significantly increased the rate of constitutive apoptosis. For the time course study R-roscovitine (20 μM) and NG75 (10 μM) increased the rate of apoptosis dramatically after 8 h, while HD at 10 μM did not increase the basal rate of apoptosis (FIG. 1B). The inventors deliberately used HD at 10 μM, a concentration that did not significantly affect the rate of constitutive apoptosis per se, as the inventors wanted to test the effect of the CDK inhibitor on delayed apoptosis induced by survival factors (see below and FIG. 1H). Systematic study of the cellular targets of R-roscovitine has revealed that it has a remarkably high specificity for CDK1, CDK2 and CDK5 and not for other kinases including CDK4 and CDK6 and in addition both NG-75 and HD have also high specificity for the same CDKs. Indeed, the crystal structure of human CDK2 complexed with R-roscovitine has been described together with evidence showing the R-stereoisomer of roscovitine is slightly more potent at inhibiting purified CDK1/cyclin B than the S-stereoisomer (IC₅₀=0.45 μM and 0.95 μM respectively). Our own studies with the stereoisomers used at 20 μM showed that rates of apoptosis assessed by annexin V binding and confirmed by morphological criteria after 6 h were 12.4±0.4% for control, 12.8±1.3% for DMSO (0.04%) control, 75.9±3.5% for R-roscovitine and 75.6±3.3% for S-roscovitine (mean±s.e.m.; n=4 separate donors each reading performed in triplicate).

Since CDK inhibitors were able to accelerate apoptosis, the inventors investigated whether these compounds could also reverse the effects of agents known to delay apoptosis through different signaling pathways (Riley, N. A. et al. Anti-inflammatory & Anti-Allergy Agents in Medicinal Chemistry 5, 3-12 (2006)). For example, dbcAMP penetrates the cell membrane to mimic endogenous cAMP and delays neutrophil apoptosis by a mechanism that does not involve a direct effect on protein synthesis and may act independently of PKA (Martin et al. J. Biol. Chem. 276, 45041-45050 (2001)). GM-CSF has been demonstrated to inhibit apoptosis through the GM-CSF receptor, leading to activation of phosphoinositide 3-kinase (PI3K), janus kinase 2 (JAK2) and signal transducer and activator of transcription 1 (STAT1). Lipopolysaccharide (LPS) binds to Toll-like receptor (TLR)4, that interacts with CD14 eventually leading to mitogen-activation kinases (MAPK), PI3K and/or NF-κB signaling to inhibit apoptosis. In addition, there is strong evidence suggesting that much of the LPS induced delay of neutrophil apoptosis is dependent on the presence of monocytes (Sabroe, I. et al. J. Immunol. 168, 4701-4710 (2002), Sabroe, I. et al. J. Immunol. 170, 5268-5275 (2003)). The effects of CDK Inhibitors on dbcAMP, GMCSF and LPS mediated survival of neutrophils was investigated and the results shown in FIG. 1(F-H). In the study, human neutrophils (5×10⁶ cells/ml) were pre-incubated with 0.2 μM dbcAMP, 50 U/ml GMCSF or 1 μg/ml LPS as indicated, in IMDM with 10% autologous serum for 30 min. Subsequently, appropriate concentrations of R-roscovitine (F), NG75 (G) or HD (H) were added and the cells incubated for a further 20 h. Apoptosis was assessed by annexin-V-FITC, and checked by morphology.

As shown in FIGS. 1 F-H, surprisingly however, the CDK inhibitors were able to over-ride all of these survival signals in a concentration-dependent manner (FIG. 1F-H) even at concentrations that did not directly induce apoptosis per se (see 10 μM HD data). This latter observation suggests that the effect of the CDK inhibitors on survival factor induced delay of apoptosis occurs independently, or is more sensitive to, any direct effects on apoptosis that they have and that the CDK inhibitors can over-ride neutrophil survival irrespective of the signaling pathways triggered by anti-apoptotic agents.

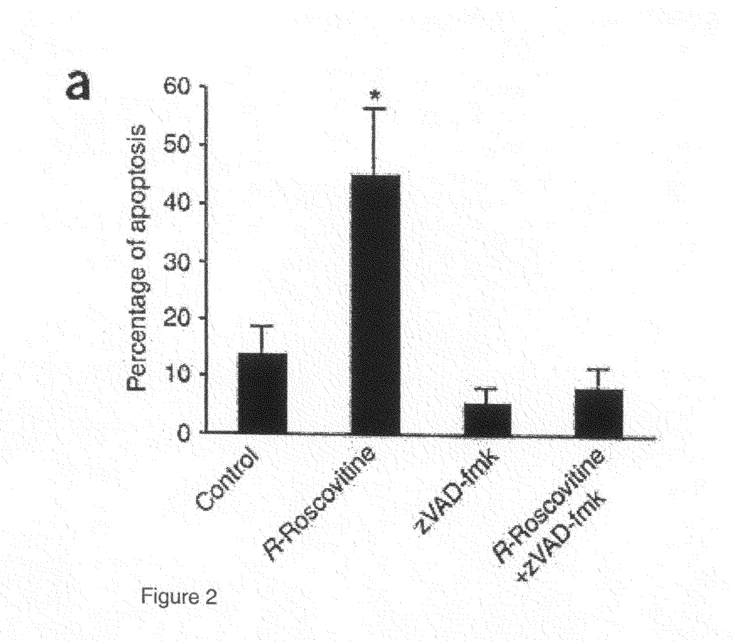

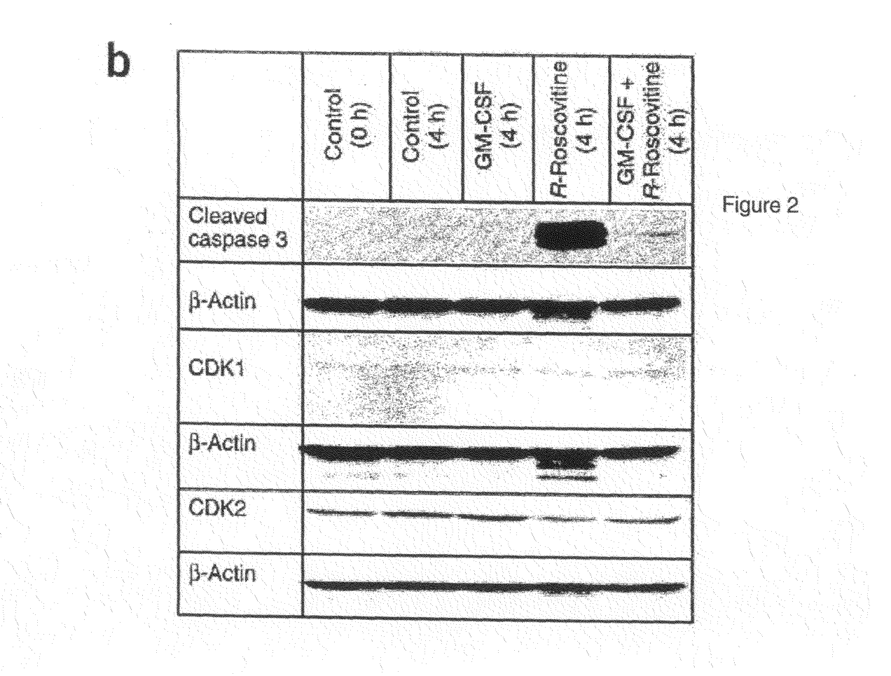

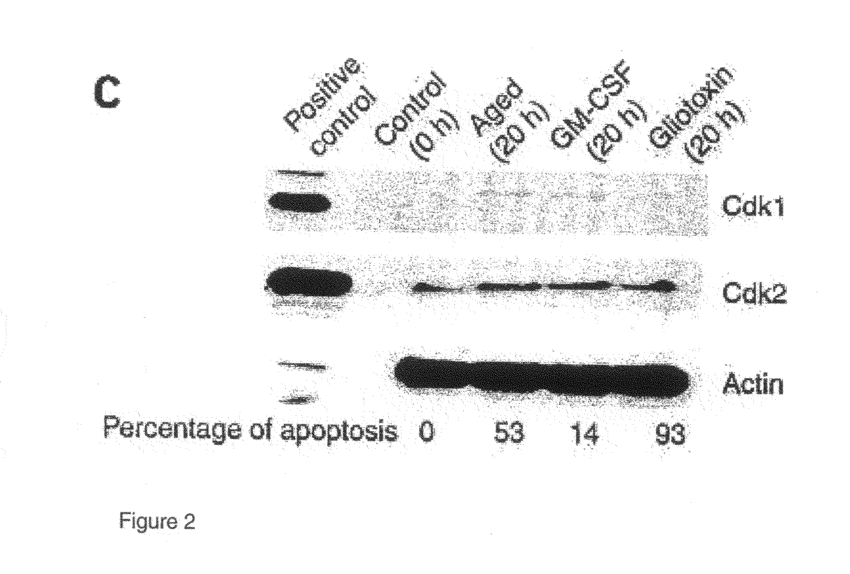

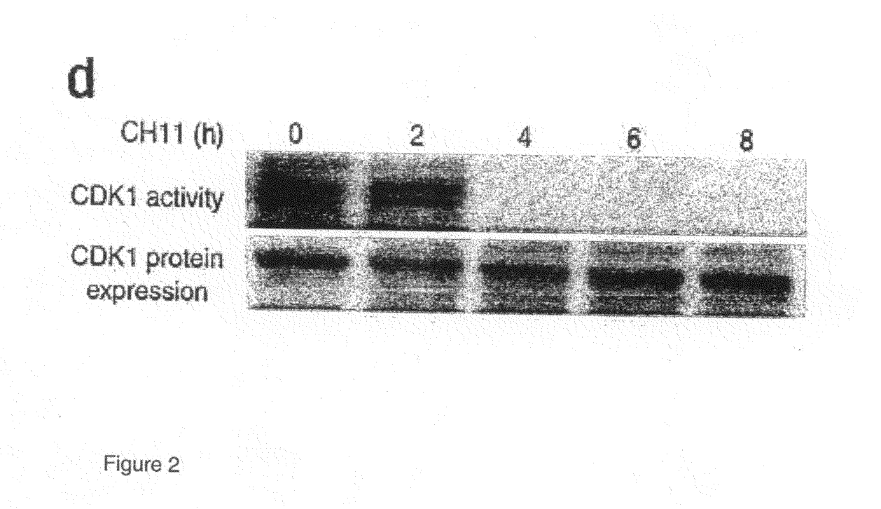

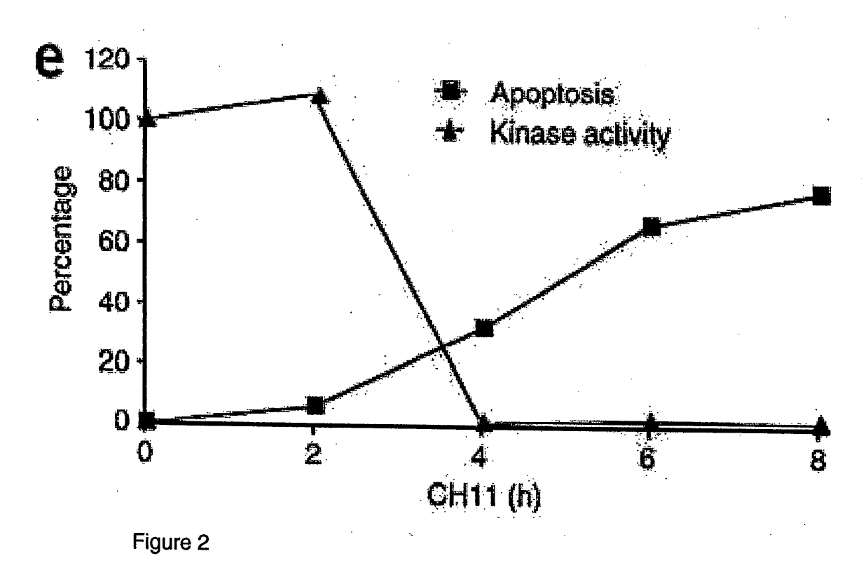

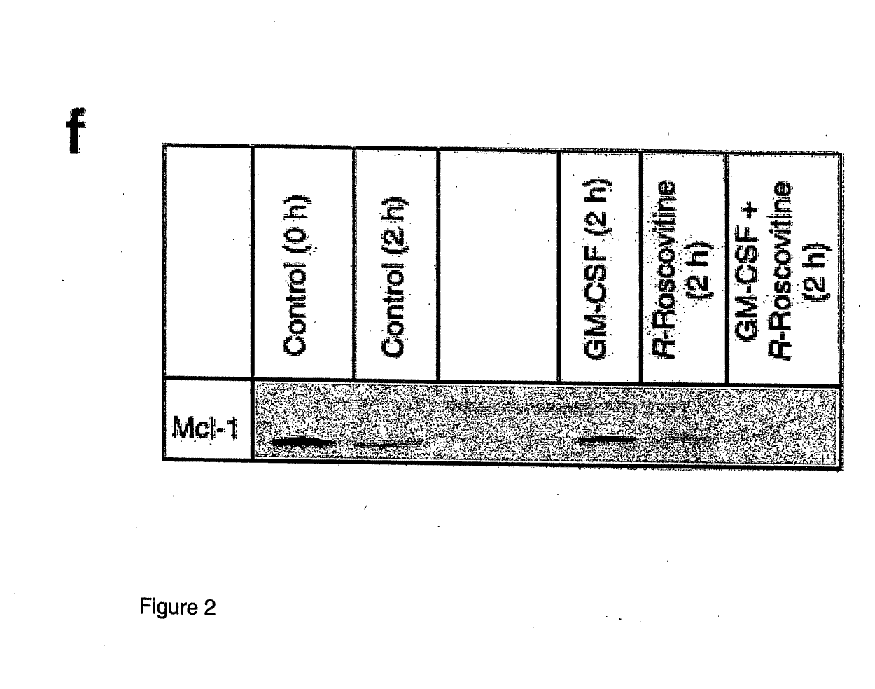

To investigate potential underlying molecular mechanisms, the inventors investigated whether the CDK inhibitor-induced apoptosis was caspase-dependent. The results are shown in FIGS. 2A and 2B. Human neutrophils (5×10⁶ cells/ml) were incubated in IMDM with 10% autologous serum for 6 h with R-roscovitine (20 μM) plus or minus zVAD-fmk (100 μM). Apoptosis was assessed by annexin-V-FITC, and checked by morphology. CDK1 and CDK2 proteins are expressed in neutrophils and R-roscovitine induces caspase cleavage in neutrophils

For (B), Human neutrophils (5×10⁶ cells/ml) were lysed after isolation (0 h) or after treatment with buffer, GM-CSF (50 U/ml), R-roscovitine (20 μM), or GM-CSF (50 U/ml) plus R-roscovitine (20 μM) for 4 h. The proteins were subsequently separated on an SDS gel. Following western blotting, PVDF membranes were probed with anti-caspase 3, anti-CDK1, anti-CDK2 or anti-β-actin antibodies. Blots are representative of at least 3 separate experiments.

Thus, the inventors have demonstrated that CDK inhibitor-induced apoptosis was caspase-dependent since pre-incubation of neutrophils with the broad-range caspase inhibitor z-VAD-fmk prevented R-roscovitine induced apoptosis (FIG. 2A). Direct verification that CDK inhibitor activates caspases in neutrophils is demonstrated in FIG. 2B where R-roscovitine results in caspase-3 cleavage as detected by western blot analysis. In addition, the inventors show that R-roscovitine induced caspase-3 cleavage is attenuated when neutrophils are co-cultured with the pro-survival factor GMCSF (FIG. 2B). At the early time point of 4 h, the rate of basal apoptosis is low (<5%; FIG. 1B) and consequently levels of caspase 3 cleavage is minimal (FIG. 2B). However, caspase-3 cleavage is already very evident at 4 h when neutrophils are treated with the R-roscovitine alone. R-roscovitine treatment in the presence of the pro-survival factor GM-CSF reduced the amount of caspase-3 cleavage (FIG. 2B) probably because of competing pro- and anti-apoptotic pathways. At 20 h however the pro-apoptotic effect of R-roscovitine clearly is dominant over survival factor mediated effects (FIG. 1F).

Since neutrophils are terminally differentiated and do not undergo cell division, CDKs and their associated partners in neutrophils have not been studied in any detail until the present. The inventors assessed CDK1 and CDK2 protein expression before and during neutrophil apoptosis. Briefly, human neutrophils (5×10⁶ cells/ml) were lysed after isolation (0 h) or after treatment with buffer, GM-CSF (50 U/ml) or gliotoxin (0.1 μg/ml) for 20 h. The proteins were subsequently separated on an SDS gel. Following western blotting, PVDF membranes were probed with anti-CDK1 or anti-CDK2 antibodies. Blots are representative of 3 separate experiments.

The results show that CDK1 and CDK2 protein expression does not change during neutrophil apoptosis Both CDK1 and CDK2 were found to be present by western blotting analysis (FIGS. 2B and C). In other cell systems CDK1 has been shown to bind to cyclin A and cyclin B, and CDK2 binds to cyclin E and E whereas CDK5 does not appear to bind to cyclins but is activated by non-cyclin p35 and p39 regulatory proteins. However the inventors' studies suggest that there does not appear to be a difference in CDK protein levels in freshly isolated neutrophils (a process that takes approximately 2 h to complete), aged neutrophils (for 4 h), or those treated with GM-CSF, R-roscovitine alone or in combination (for 4 h). Even when neutrophils were incubated for 20 h with buffer, GM-CSF or following induction of apoptosis with gliotoxin (Ward, C. et al. J. Biol. Chem. 274, 4309-4318 (1999)) levels of the CDK did not change (FIG. 2C). Thus although CDKs appeared to be important for inducing apoptosis, CDK1 and/or CDK2 were not targets for degradation during apoptosis. Furthermore, as R-roscovitine can also inhibit CDK5 the inventors performed western blot analysis and showed CDK5 presence in human neutrophils (data not shown). This finding is in agreement with Rosales et al. CDK activity was measured with the results shown in FIGS. 2D and E for CDK1. Neutrophils (5×10⁶ cells/ml) were treated with anti-Fas activating antibody CH11 (500 ng/ml) for the indicated time. Subsequently, 10⁷ cells were lysed for each sample, the lysate incubated with anti-CDK1 antibody and CDK1 activity measured by ³²P transfer to histone H1. Apoptosis (annexin-V-FITC/PI) and CDK1 activity were measured every 2 h over 8 h. CDK1 activity was detected at time 0 h and 2 h but subsequently became undetectable as the levels of apoptosis Increased over time. Again, no difference in CDK1 expression levels could be detected during the experiment. CDK2 activity was also measured but no activity could be detected (results not shown). Results are representative of at least 3 separate experiments. Interestingly, CDK1 (FIGS. 2D and E), CDK2 (not shown) and CDK5 activity was demonstrated in neutrophils. CDK1 activity in isolated neutrophils decreased rapidly as the cells underwent apoptosis induced by the anti-Fas activating antibody CH11 (FIG. 2E).

The inventors further probed the effect of R-roscovitine on the expression of a key survival protein Mcl-1 shown to be a key regulator of apoptosis (Michels et al, Int. J. Biochem. Cell Biol. 37, 267-271 (2005), Moulding et al, Blood 92, 2495-2502 (1998)). Human neutrophils (5×10⁶ cells/ml) were treated with buffer, GM-CSF (50 U/ml), R-roscovitine (20 μM), or GM-CSF (50 U/ml) plus R-roscovitine (20 μM) for 4 h. The samples were subsequently lysed and the proteins separated on an SDS gel. Following western blotting, PVDF membranes were probed with anti-Mcl-1 antibody. Blots are representative of at least 3 separate experiments. The results are shown in FIG. 2F. The inventors demonstrate that levels of Mcl-1 in isolated cells falls rapidly within 2 h of culture, an effect that is prevented by GM-CSF treatment. R-roscovitine inhibits GM-CSF mediated up-regulation of Mcl-1 (FIG. 2F).

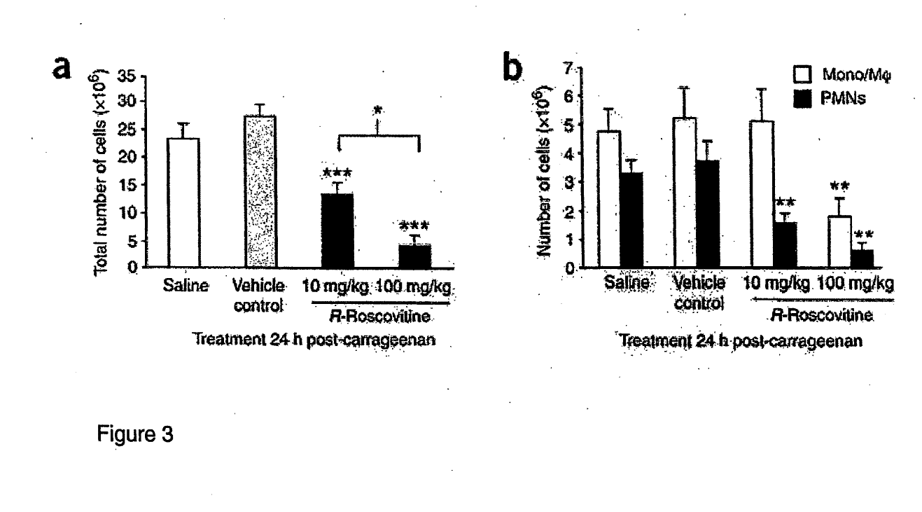

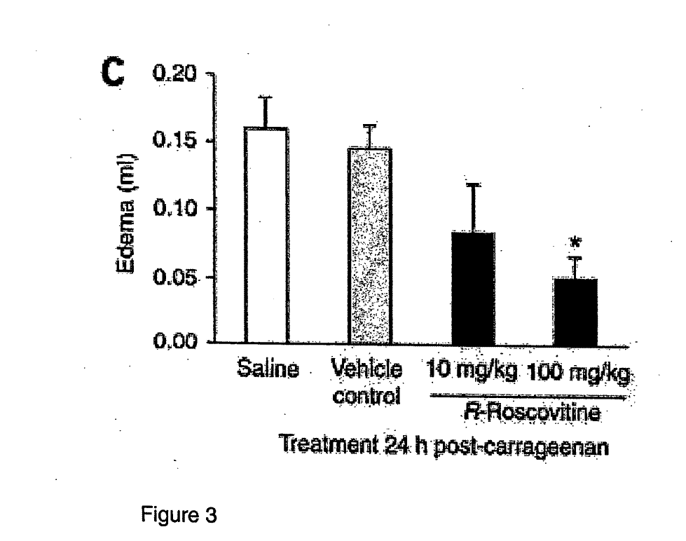

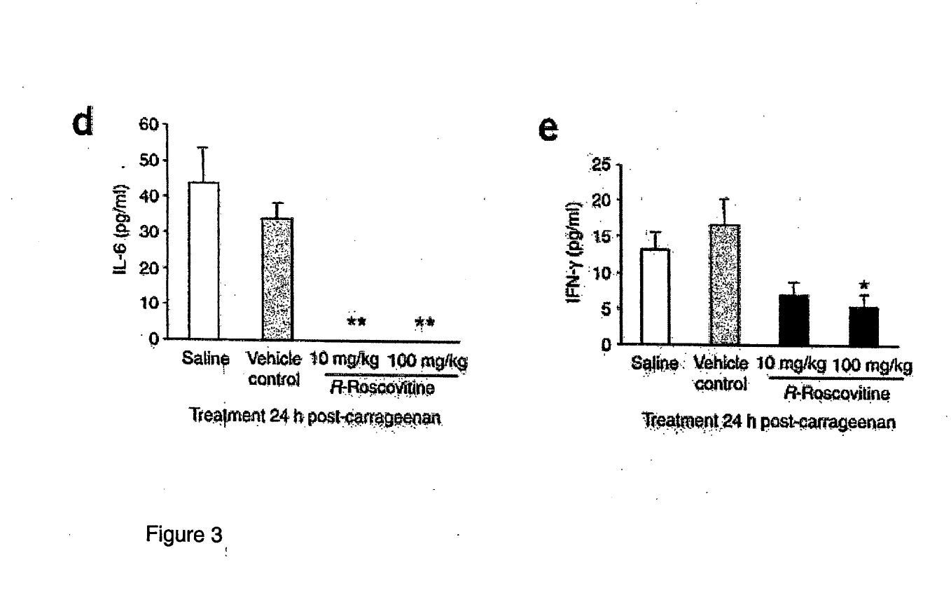

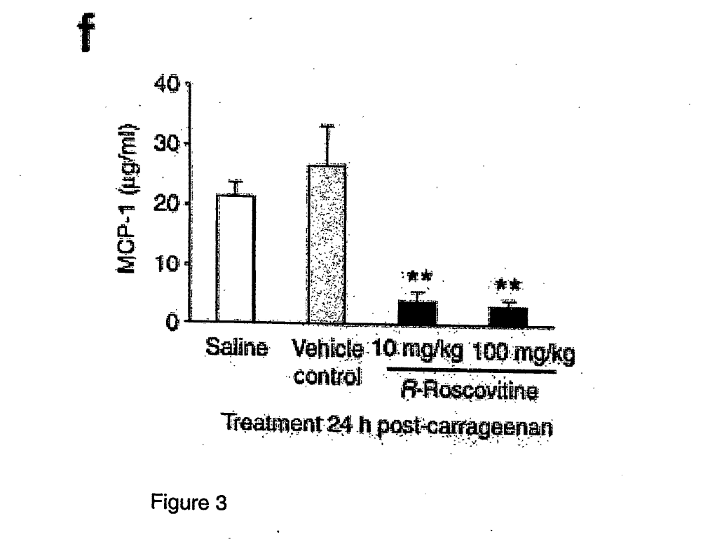

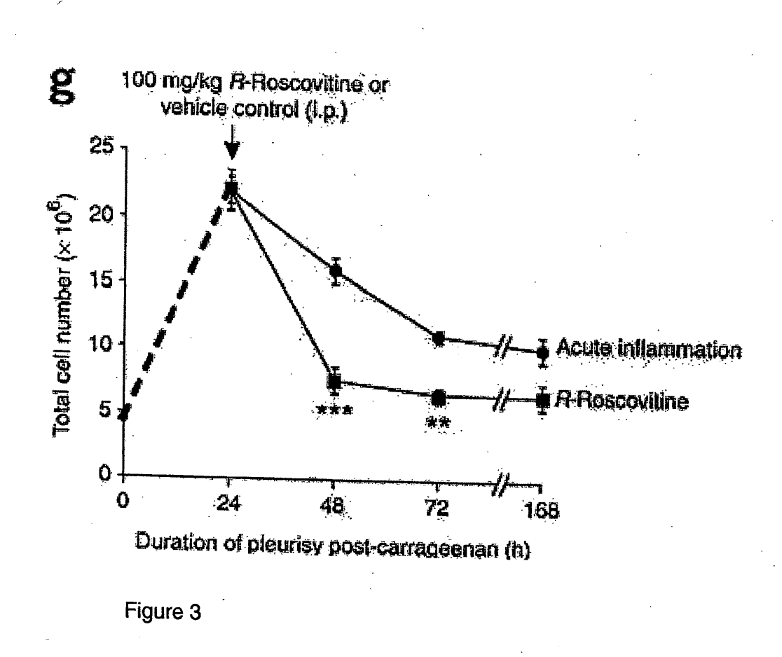

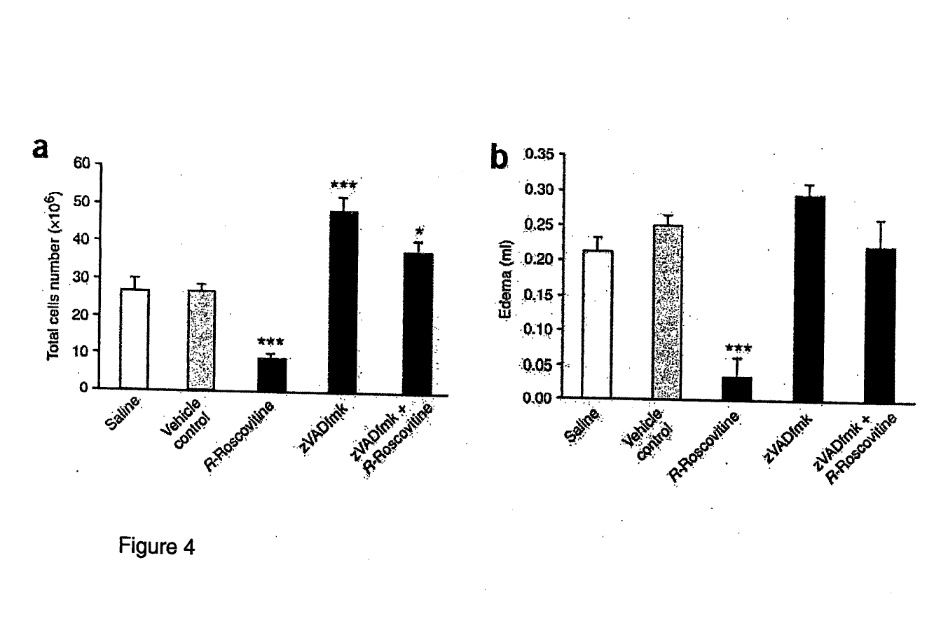

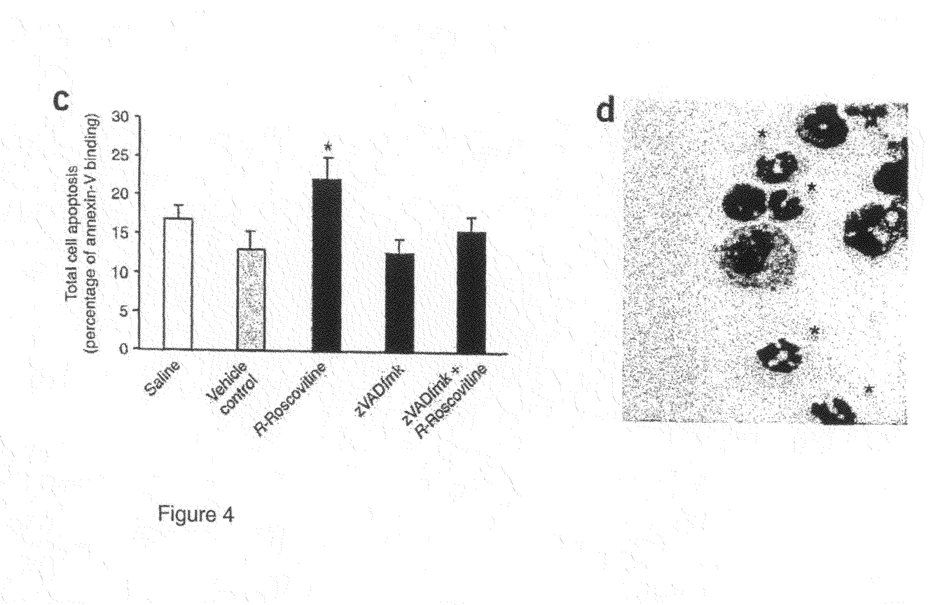

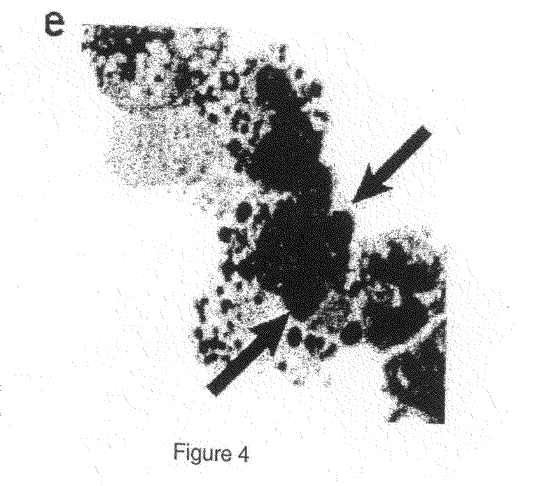

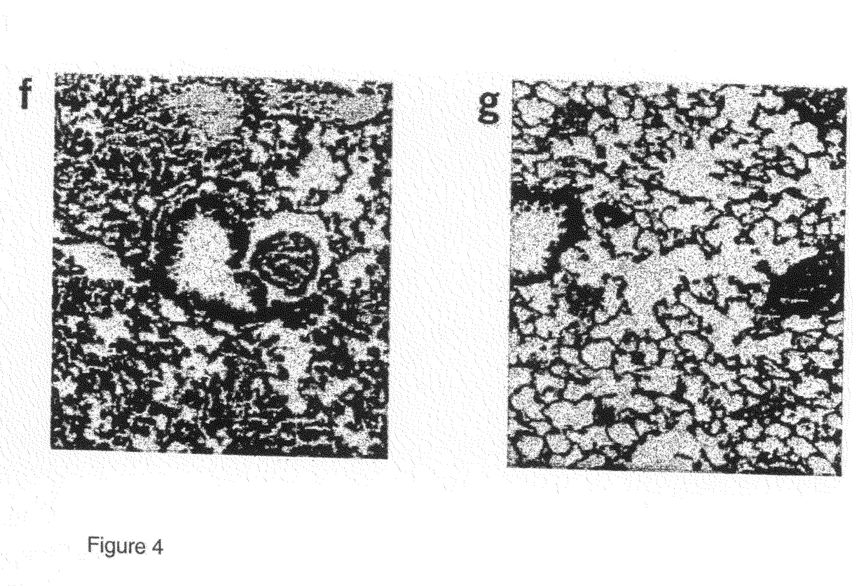

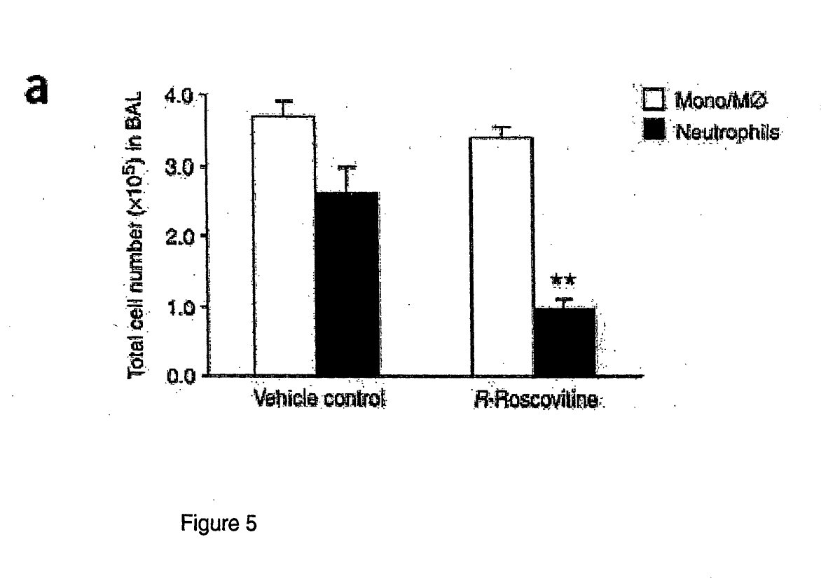

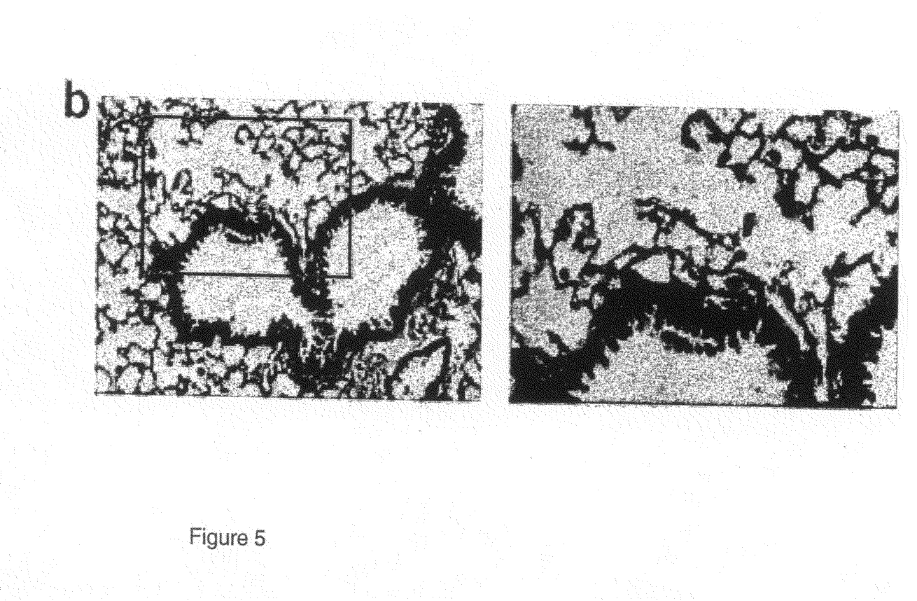

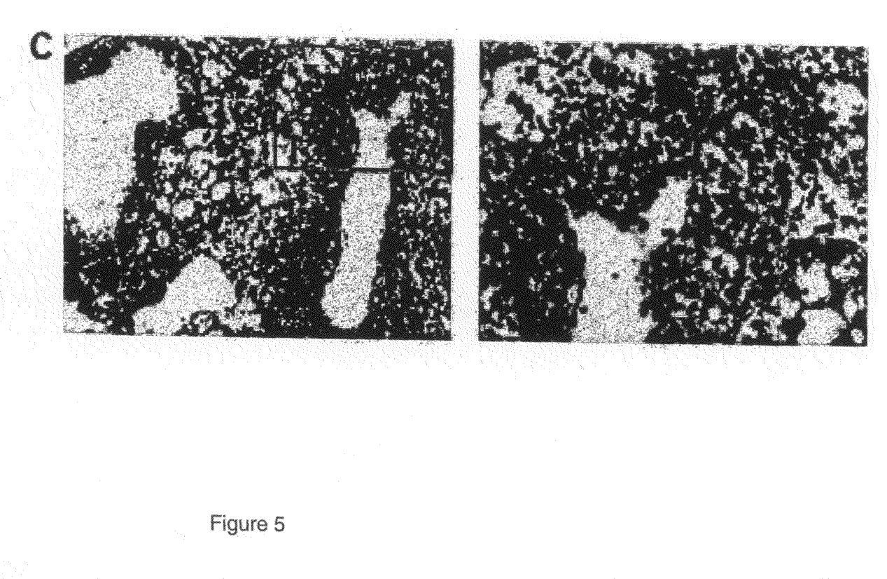

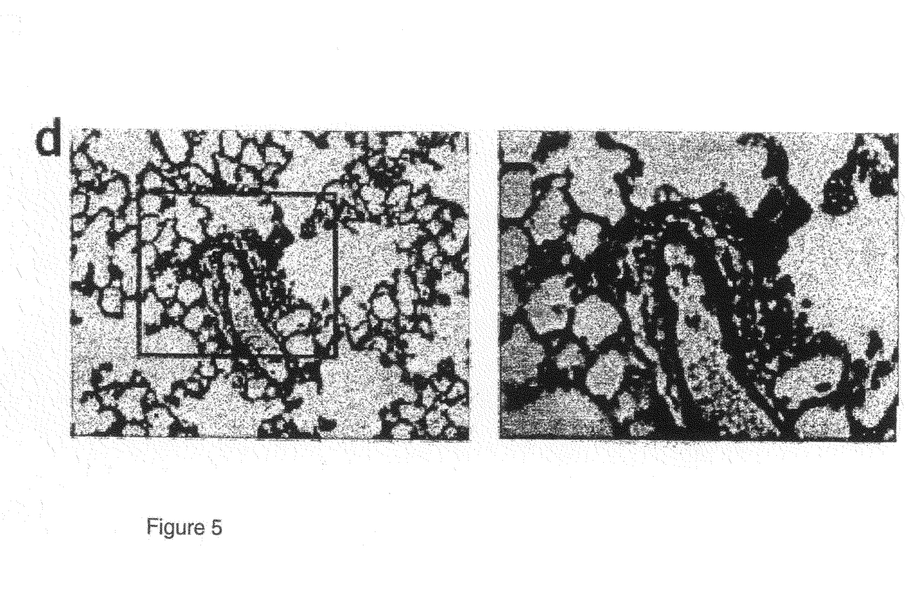

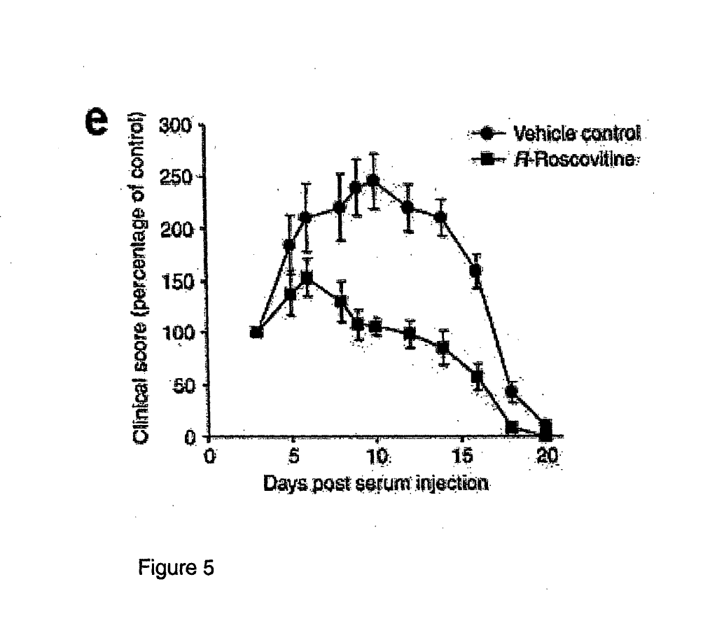

Having shown in vitro that neutrophil apoptosis was dramatically influenced by CDK inhibitors the inventors next determined the effects of R-roscovitine on resolution of neutrophil-dominant inflammation in vivo. An acute resolving model of carrageenan-induced pleural inflammation was used to assess the effects of the CDK inhibitor. This well established model has been used successfully to examine the kinetics of inflammatory cell recruitment and to investigate inflammatory mechanisms as well as pharmacological agents with potential anti-inflammatory properties (Gilroy, D. W. et al. Nat. Med. 5, 698-701 (1999), Gilroy, D. W. et al. FASEB J. 17, 2269-2271 (2003), Sawatzky et al. Am. J. Pathol. 168, 33-41 (2006), Cailhier, J. F. et al. Am. J. Respir. Crit. Care Med. 173, 540-547 (2006)). In the present study, 24 h following intra-pleural injection of 1% carrageenan, male C57/bl6 mice were treated with saline, DMSO, 10 or 100 mg/Kg R-roscovitine (i.p.). All mice were killed 36 h post-carrageenan and the number of total pleural inflammatory cell numbers (FIG. 3A), mononuclear cells and PMNs numbers (FIG. 3B) and exudate volumes (FIG. 3C) were measured. Values represent the mean±s.e.m. of 8-10 mice per group. **p<0.01 and ***p<0.001 denotes significant difference from DMSO control.

R-roscovitine accelerated the resolution of established inflammation when administered i.p. 24 h after intra-pleural injection of 1% carrageenan (FIG. 3). Thus, 10 mg/kg R-roscovitine (i.p.) treatment inhibited the total inflammatory cell number by greater than 50% compared with vehicle control (FIG. 3A), with a reduction in the number of monocytes/macrophages and neutrophils (FIG. 3B). Of note, 100 mg/kg R-roscovitine reduced the amount of inflammatory cells to near baseline levels that is normally found in the naïve murine pleural cavity. R-roscovitine also demonstrated functional anti-inflammatory effects since edema formation, measured by the total exudate volume obtained by pleural wash-outs, decreased by three-fold with R-roscovitine treatment compared with saline control (*p<0.05; FIG. 3C).

In parallel, release of pro-inflammatory mediators IL-6 (FIG. 3D), IFN-γ (FIG. 3E), and MCP-1 (FIG. 3F) in the inflammatory exudate was also diminished in the presence of R-roscovitine. In this study, 24 h following intra-pleural injection of 1% carrageenan, male C57/bl6 mice were treated with saline, DMSO, 10 or 100 mg/Kg R-roscovitine (i.p.). 36 h post-carrageenan, IL-6 (D), IFN-γ (E) and MCP-1 (F) were measured in pleural exudates with a cytokine bead assay. Values represent the mean±s.e.m. of 8-10 mice per group. **p<0.01 and *p<0.001 denotes significant difference from DMSO control.

Having demonstrated a concentration-dependent effect of R-roscovitine on accelerating inflammation resolution, the inventors next determined the effect of the CDK inhibitor over a period of seven days. R-roscovitine (100 mg/kg i.p.) administered at the peak of inflammation (at 24 h) resulted in marked resolution of inflammation, as assessed by pleural cavity total cell numbers at all time points examined (FIG. 3G) and total cell, neutrophil and monocytes/macrophages numbers counted 36 h post carrageenan (FIGS. 3A and B) providing further evidence that the CDK inhibitor does enhance inflammatory resolution. Since R-roscovitine induced caspase-dependent apoptosis of human neutrophils in vitro (FIGS. 2A and B), it was important to establish whether the enhanced resolution of inflammation observed with systemic R-roscovitine was mediated by a similar induction of caspase-dependent inflammatory cell apoptosis in vivo. To establish this, 24 h following intra-pleural injection of 1% carrageenan, male C57/bl6 mice were treated i.p. with 10 mg/Kg R-roscovitine (i.p.) and/or 1 μg/Kg zVAD-fmk (i.p. at 4 h intervals). All mice were killed 36 h post-carrageenan and total pleural inflammatory cell numbers (A) and exudate volumes (B) were measured. The effect of zVAD-fmk was studied as follows (FIG. 4C): 24 h following intra-pleural injection of 1% carrageenan, male C57/bl6 mice were treated i.p. with 10 mg/Kg R-roscovitine (i.p.) and/or 1 μg/Kg zVAD-fmk (i.p. at 4 h intervals). All mice were killed 36 h post-carrageenan and apoptosis was analyzed in pleural inflammatory cells by annexin V labeling and flow cytometry. Inflammatory cell apoptosis was also assessed by morphology on haematoxylin and eosin stained cyto-centrifuge preparations.

Systemic administration of the broad-spectrum caspase inhibitor zVAD-fmk prevented the R-roscovitine-induced decrease in inflammatory cells and edema formation in the pleural cavity in the acute model of carrageenan-induced pleural inflammation (FIG. 4). Interestingly, zVAD-fmk, prevented the resolution of inflammation in vivo, by increasing the total inflammatory cell number (FIG. 4A) and edema formation (FIG. 4B) when compared with saline or vehicle controls. Furthermore, the increase in inflammatory cell apoptosis (FIG. 4C) and macrophages containing apoptotic bodies (see FIGS. 4D and E) observed with R-roscovitine treatment in the pleural cavity was inhibited dramatically by treatment with zVAD-fmk. The inventors also noted that the lungs of the mice treated with carrageenan in the pleural cavity also had a consequent marked inflammatory infiltrate in the lung sections (FIG. 4F). This lung inflammation was also dramatically reduced by R-roscovitine treatment (FIG. 4G).

The anti-inflammatory and pro-resolving effects with R-roscovitine treatment were further confirmed in another two inflammatory models. The effect of the CDK inhibitor R-roscovitine on the resolution of bleomycin-induced lung inflammation was assessed as follows, the results being shown in FIG. 5A: 100 mg/Kg R-roscovitine (i.p.) was given 48 h following intra-tracheal administration of bleomycin where neutrophil (closed bars) and monocyte/macrophage (open bars) numbers were established by differential cell counts obtained from BAL fluid three days post-bleomycin administration.

In the chronic bleomycin-induced lung injury model, R-roscovitine (100 mg/kg i.p.), given after inflammation was established in the lung, reduced the total number of neutrophils in the bronchioalveolar lavage (BAL) fluid 3 days post-bleomycin administration (**p<0.01; FIG. 5A). The effect of R-roscovitine on inflammation and tissue damage in this model was established as follows: 100 mg/Kg R-roscovitine (i.p.; D) or vehicle control (0.5% DMSO; i.p.; C) was given 48 h following intra-tracheal administration of bleomycin or saline (sham control; B). Seven days post-bleomycin treatment, lung inflammation and histological tissue damage was assessed by morphological examination (original magnification×40) of haematoxylin- and eosin-stained paraformaldehyde-fixed lung sections for each treatment group. The results are shown in FIGS. 5B-D

Histological examination showed that when the bleomycin-induced lung injury was allowed to persist for seven days, R-roscovitine prevented bleomycin induced tissue damage (FIG. 5B-D). R-roscovitine, when administered with the sham intra-tracheal saline control, had no obvious detrimental effects on the lung pathology (FIG. 5B).

In another set of experiments the inventors investigated the effects of roscovitine on bleomycin-induced lethality. By day 12, 3 out of 8 mice treated with bleomycin alone had died whereas none of the control and roscovitine treated mice had died. By day 13, one of the bleomycin and roscovitine treated animals had died before terminating the experiment on day 14 (data not shown).

Further, the inventors further investigated the effect of R-roscovitine in a model of passively-induced arthritis. Mice (n=10 in each group) were injected twice (days 0 and 3) with K/B×N serum derived from arthritic (day 60) K/B×N transgenic mice. Clinical scores were assessed on the days indicated. Control mice received vehicle 0.5 ml i.p.) and treated mice received R-roscovitine (0.5 ml, 10 mg/kg, i.p.) on days 3, 5, 7, 9, 11, 13, 15, 17, 19. The results are shown in FIG. 5E. Data are expressed as the percentage of the clinical score obtained on day 3 before the first injection of R-roscovitine. the inventors observed that arthritis as assessed by clinical scores dramatically resolved quicker in the roscovitine than the control treated animals.

### Discussion

Specific induction of inflammatory cell apoptosis represents a new approach in the future treatment of inflammatory diseases. The coordinated cascade of events that occurs following inflammatory insult, either in an acute or chronic setting, is highly regulated from onset to the resolution phase. This involves a series of checkpoints that control cell migration and infiltration, survival and perpetuation of the inflammatory response through the generation of many mediators (e.g., PGs, PAF, leukotrienes, lipoxins), growth factors (e.g., GM-CSF), cytokines (e.g., TNFα, IL-1β) and chemokines (e.g., IL-8, eotaxin). The life span of migrated cells is also highly regulated and each individual cell is susceptible to die in a pre-programmed manner via apoptosis, or may persist in situ in a potentially detrimental fashion to promote tissue destruction via continued excessive secretory activity or through the consequences of cell death by secondary necrosis. CDKs are critical cell signaling proteins that traditionally have been thought to exclusively control the fate of proliferating cells, where CDK dysfunction is likely involved in increased cell turnover and tumor progression. Current therapies aimed at inhibiting CDKs are being developed for the treatment of various cancers, such as non-small cell lung cancer and breast cancer.

As noted herein, the present inventors have surprisingly shown that CDK inhibitors promote apoptosis in neutrophils, considered to be terminally differentiated cells.

Inflammatory cell turnover at sites of inflammation is kept in check by the balance between cell recruitment, apoptosis and their subsequent clearance. Furthermore, the ingestion of apoptotic cells by inflammatory macrophages also promotes the synthesis and release of mediators with anti-inflammatory properties (e.g., TGF-β1 and IL-10). The present study has revealed a novel mechanism for accelerating apoptosis of human neutrophils by CDK inhibitors in vitro and demonstrated that this enhancement of apoptosis in vivo results in increased resolution of neutrophil-dependent inflammation in mice. The inventors have shown that human neutrophils possess CDK1 (FIG. 2), CDK2 (FIG. 2) and CDK5 (data not shown), which are likely to play a functional role in inflammation. The inventors have also demonstrated that the specific inhibitors of CDKs, R-roscovitine, NG75 and HD induce human neutrophil apoptosis in a time- and concentration-dependent manner involving a caspase-dependent mechanism. Importantly, the CDK inhibitors induced apoptosis even in the presence of powerful pro-survival agents (e.g. dbcAMP, GM-CSF and LPS). This is of particular interest since the pro-survival agents selected for testing in this study retard human neutrophil apoptosis by separate and distinct molecular mechanisms. There is compelling evidence suggesting that contaminating monocytes contribute to LPS mediated neutrophil survival. LPS may thus trigger monocytes to synthesis and release other survival factors (e.g., IL-1, GM-CSF, IL-8, etc) to induce neutrophil survival. Whatever the precise mechanisms of action of LPS on neutrophils apoptosis, the inventors' data clearly show that the CDK inhibitors can overcome them. Thus, the ability of CDK inhibitors to override endogenous pro-survival mediators further suggests their potential use in inflammatory diseases where levels of such mediators are found elevated locally at inflammatory sites. The inventors found that although protein expression levels of CDK1 and CDK2 enzymes did not change during the apoptotic process their enzymatic activity was altered. Thus, treatment of neutrophils with the pro-apoptotic anti-Fas antibody CH11 resulted in loss of CDK1 activity prior to the onset of apoptosis. In contrast, a previous study has shown that non-cycling neuronal cells require CDK activation for the induction of apoptosis and CDK inhibitors are able to prevent cell death.

In distinct contrast, the inventors' results suggest that CDK activity is necessary and fundamental to neutrophil survival. Furthermore, the inventors show that R-roscovitine induced apoptosis is caspase dependent and is associated with a loss of Mcl-1 expression; a member of the Bcl-2 family of anti-apoptotic proteins known to be important in controlling neutrophil apoptosis (Moulding. Blood 92, 2495-2502 (1998)). The loss of Mcl-1 expression by CDK inhibitors has been the focus of much interest and is likely to be an important mechanism involved in controlling apoptosis. In addition, since CDK5 is also a target for R-roscovitine and this CDK has been reported in human neutrophils (Rosales, J. L et al. Biol. Chem. 279, 53932-53936 (2004)), the inventors performed western blot analysis and confirmed that CDK5 protein is indeed present in human neutrophils (data not shown).

Having demonstrated that CDK inhibitors can regulate apoptosis in vitro, the inventors proceeded to confirm that the R-roscovitine promoted in a dose-dependent fashion the resolution of inflammation in an in vivo model of carrageenan-induced pleurisy. R-roscovitine also inhibited the release of pro-inflammatory cytokines, including IL-6, MCP-1 and IFN-γ, further supporting a pro-resolution role. Importantly, it was demonstrated that R-roscovitine induced apoptosis in vivo assessed by annexin V labeling and through morphological analysis. The inventors have previously shown that inhibiting pro-survival molecules from the MAPK and Bcl-2 families, namely Bcl-_{xL} and ERK1/2, can promote the resolution of inflammation by inducing inflammatory cell apoptosis in an acute in vivo model of carrageenan-induced pleurisy in the rat (Sawatzky et al. Am. J. Pathol. 168, 33-41 (2006)). Conversely, the inventors have also demonstrated that inhibition of the pro-apoptotic molecule, Bax, prevents the resolution of inflammation in this model. Thus, carrageenan-induced pleurisy appears to be an excellent model for pharmacological manipulation of apoptosis in order to investigate inflammatory resolution.

Furthermore, R-roscovitine given at the height of the inflammatory response enhanced the resolution of bleomycin induced lung inflammation. This model has been used not only for its clinical relevance (Azambuja et al Pulm. Pharmacol. Ther. 18, 363-366 (2005)) but also because the progression of the dramatic acute inflammatory response leads to chronic inflammation and fibrosis (Nagase, T. et al. Nat. Med. 8, 480-484 (2002), Teder, P. et al. Science 296, 155-158 (2002)). In this model the inventors show that the early neutrophil accumulation into bronchioalveolar lavage fluid and that the later lung inflammation and injury is attenuated by R-roscovitine treatment. Importantly, the inventors also observed that R-roscovitine reduced bleomycin-induced lethality indicating that the bleomycin-induced prolonged inflammation leading to lung damage and consequent death is also attenuated by the CDK inhibitor. In another inflammatory model, the inventors observed a marked enhancement of the resolution of the inflammatory response in serum-induced arthritis as assessed by improved clinical scores. The inventors' in vitro work showing that the CDK inhibitors promote neutrophil apoptosis is of particular interest and adds to the body of evidence indicating that neutrophils are critically important in regulating the inflammatory responses in viva, including models of carrageenan-induced pleurisy (Sawatzky et al. Am. J. Pathol. 168, 33-41 (2006)), bleomycin induced-lung injury (Nagase, T. et al. Nat. Med. 8, 480-484 (2002), Teder, P. et al. Science 296, 155-158 (2002)) and arthritis (Jonsson, H. Nat. Med. 11, 666-671 (2005)).

The inventors have also investigated whether the enhanced resolution of inflammation by CDK inhibitors is mediated by enhanced apoptosis in vivo. The inventors have shown that inhibition of apoptosis with the broad-spectrum caspase inhibitor, zVAD-fmk, prevents the resolution of inflammation in the murine model of carrageenan-induced pleurisy. The dosing regime for zVAD-fmk administration is paramount to elicit an effect in vivo and was based on previous research where injections were administered every 3 h (De Paepe et al, Am. J. Physiol Lung Cell Mol. Physiol 287, L730-L742 (2004)). Importantly, R-roscovitine-induced apoptosis and anti-inflammatory effects can be reversed in vivo by the caspase inhibitor zVAD-fmk, providing further evidence that the anti-inflammatory mechanism of the CDK inhibitor is due to the induction of caspase-dependent inflammatory cell apoptosis.

CDKs are abundant in many cell types and critically regulate cell division and proliferation. Here the inventors demonstrate that systemic administration of a specific inhibitor of CDKs is able to induce apoptosis of inflammatory cells in situ and promote inflammatory resolution. Other murine in vivo studies investigating the effect of CDK inhibition in cancer have shown that systemic administration of R-roscovitine caused a specific reduction in tumor size (McClue, S. J. et al. Int. J. Cancer 102, 463-468 (2002)). The authors of that study also noted that R-roscovitine (even with high doses of 2 g/kg) is well tolerated. It is widely acknowledged that tumor cells have defective CDK pathways, which promote cell proliferation and prevent cell apoptosis (Vermoulen, K et al. Cell Prolif. 36, 131-149 (2003), Senderowicz, A. M. Oncogene 22, 6609-6620 (2003)), it is therefore thought that CDK inhibitors specifically target this to elicit their therapeutic action. Our study is the first to demonstrate that CDK inhibitors also promote apoptosis in non-proliferating inflammatory cells in vitro and accelerate inflammatory resolution by promoting apoptosis and subsequent safe clearance of neutrophils by macrophages in vivo. In conclusion the inventors have identified a novel role of the CDK inhibitors that may have potential for the treatment of diseases associated with increased or persistent inflammatory responses.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A method of inducing and/or accelerating apoptosis of granulocytes, said method comprising administering to said granulocytes an effective dose of a CDK inhibitor.

2. A method of treatment of granulocyte mediated disorder or disease in an individual, wherein said method comprises administration of a CDK inhibitor.

3. The method according to claim 2 wherein the disease or disorder is an inflammatory disease or disorder.

4. The method according to claim 2 wherein the disease or disorder is a non-neoplastic inflammatory disease.

5. The method according to claim 2, wherein said treatment is treatment of a non-proliferative stage of an inflammatory disease.

6. The method according to claim 3, wherein said disease or disorder is a chronic inflammatory disease or disorder.

7. The method according to claim 3, wherein said disease or disorder is an acute inflammatory disease or disorder.

8. The method according to claim 3, wherein said disease or disorder is a respiratory disease or disorder.

9. The method according to claim 8, wherein the disease or disorder is selected from the group consisting of interstitial lung disease, pleurisy, asthma, chronic obstructive pulmonary disease (COPD), and acute respiratory disease.

10. The method according to claim 9, wherein said interstitial lung disease is acute interstitial pneumonia or idiopathic pulmonary fibrosis.

11. The method according to claim 2, wherein said disease or disorder is arthritis.

12. The method of claim 2, wherein the disease or disorder is an allergic disorder.

13. The method according to claim 2, wherein the CDK inhibitor is an inhibitor of CDK1 and/or CDK2.

14. The method according to claim 2, wherein the CDK inhibitor is an inhibitor of CDK5.

15. The method according to claim 2, wherein the CDK inhibitor is a pyrimidine or purine based inhibitor.

16. The method according to claim 2, wherein said CDK inhibitor is selected from the group consisting of roscovitine, NG75 and hymenialdisine.

17.-27. (canceled)

28. The method according to claim 1, wherein the CDK inhibitor is an inhibitor of CDK1 and/or CDK2.

29. The method according to claim 1, wherein the CDK inhibitor is an inhibitor of CDK5.

30. The method according to claim 1, wherein the CDK inhibitor is a pyrimidine or purine based inhibitor.

31. The method according to claim 1, wherein said CDK inhibitor is selected from the group consisting of roscovitine, NG75 and hymenialdisine.
